(19) United States
(12) Patent Application Publication
(10) Pub. No.: US 2020/0331935 A1
(43) Pub. Date: Oct. 22, 2020

(54) HYDROLYTICALLY LABILE SILYL ENOL ETHER FRAGRANCE KETONES OR ALDEHYDES

(71) Applicant: Henkel AG & Co. KGaA, Duesseldorf (DE)

(72) Inventors: Sascha Wilhelm SCHAEFER, Mettmann (DE); Silvia SAUF, Velbert (DE); Benjamin William BERNTSSON, Duesseldorf (DE)

(21) Appl. No.: 16/753,354

(22) PCT Filed: Sep. 25, 2018
(86) PCT No.: PCT/EP2018/075892

§ 371 Date: Apr. 3, 2020

(30) Foreign Application Priority Data
Oct. 6, 2017 (DE) 10 2017 123 282.6

(51) Int. Cl. C07F 7/18; A61K 8/58; A61Q 13/00; C11D 3/50; A61L 9/01

(52) U.S. Cl. CPC C07F 7/1804; A61K 8/585; A61L 9/01; C11D 3/50; A61Q 13/00

(57) ABSTRACT

Silyl enol ethers are disclosed that contain odoriferous ketone or aldehyde residues and are suitable for fragrancing laundry. Compositions may include the silyl enol ethers and an agent that is a washing agent, a cleaning agent, a cosmetic agent, an air care agent, an insect repellent, or combinations thereof. Silyl enol ethers release ketones and aldehydes during hydrolysis. The silyl enol ethers may have the following formula (I):

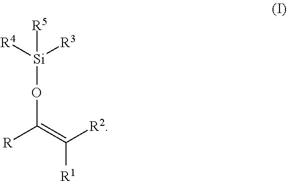

### CROSS-REFERENCE TO RELATED APPLICATIONS

The present application is a national stage entry according to 35 U.S.C. § 371 of PCT application No.: PCT/EP2018/075892 filed on Sep. 25, 2018; which claims priority to German Patent Application Serial No.: 10 2017 123 282.6, which was filed on Oct. 6, 2017; which is incorporated herein by reference in its entirety and for all purposes.

### TECHNICAL FIELD

The present disclosure relates to silyl enol ethers which contain functional groups of odorant ketones or aldehydes and which are suitable for fragrancing laundry, for example, since they release the ketones or aldehydes during hydrolysis.

### BACKGROUND

The controlled release of fragrances in the field of washing and cleaning agents in order to intensively fragrance both the product and the washing and cleaning solution and the articles treated therewith in a long-lasting manner is known in the prior art. In addition to the methods of applying fragrances to carrier materials and coating the fragranced carriers, or encapsulating fragrances or storing said fragrances in compounds, there is the possibility of chemically bonding the fragrances to carrier media, the chemical bond being slowly broken and the perfume being released.

In the prior art it is known to bond fragrant alcohols to non-volatile siloxanes, from which they are slowly released by hydrolysis. For example, WO 01/068037 A2 and GB 2319527 A describe mixtures of oligomeric silicic acid esters which contain functional groups of fragrant alcohols and are suitable, for example, for fragrancing washing and cleaning agents. Further polymeric siloxanes which are used as fragrance storage substances for alcohols, carbonyls, unsaturated ketones and aldehydes are described, for example, in EP 1716159 B1 and WO 2016/091815 A1.

Silyl enol ethers of fragrance aldehydes and ketones are described, for example, in DE 10 2013 226 098 A1. The problem addressed by this application was that of providing alternative silyl enol ether-based precursors of odorants which permit long-lasting release of the odorants and use low-molecular anchor groups which optionally also impart adhesion to the surfaces to be fragranced, such as textile surfaces.

### SUMMARY

The inventors have now surprisingly found that compounds of this kind can be prepared by utilizing the keto-enol tautomerism of odorant ketones and aldehydes, the cleavage of which during or after application then reproduces the aldo or keto form by means of retautomerization. Although the keto-enol tautomerism is well known, the equilibrium in non-further functionalized ketones and aldehydes is usually very much on the side of the carbonyl compound. However, the inventors have now found that, by trapping the enol form and converting it into a storage-stable enol-silicon compound, the enol can be removed from equilibrium such that the complete ketone or aldehyde is ultimately converted into the corresponding silyl enol ether form.

In a first aspect, a compound may include silyl enol ethers of the formula

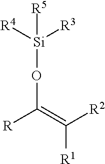

wherein
R, R¹ and R² are independently selected from H, straight-chain or branched, saturated or unsaturated, substituted or unsubstituted hydrocarbon functional groups having 1 to 20 carbon atoms and optionally up to 6 heteroatoms, such as linear or branched alkyl, alkenyl or alkynyl having up to 20, such as up to 12, carbon atoms, substituted or unsubstituted, linear or branched heteroalkyl, heteroalkenyl or heteroalkynyl having up to 20, such as up to 12, carbon atoms, and 1 to 6, such as 1 to 4, heteroatoms selected from O, S and N, substituted or unsubstituted aryl having up to 20, such as up to 12, carbon atoms, substituted or unsubstituted heteroaryl having up to 20, such as up to 12, carbon atoms, and 1 to 6, such as 1 to 4, heteroatoms selected from O, S and N, cycloalkyl or cycloalkenyl having up to 20, such as up to 12, carbon atoms, and heterocycloalkyl or heterocycloalkenyl having up to 20, such as up to 12, carbon atoms, and 1 to 6, such as 1 to 4, heteroatoms selected from O, S and N, or R and R¹ or R and R² can be combined with one another in order to form a cyclic group selected from substituted or unsubstituted aryl having up to 20, such as up to 12, carbon atoms, substituted or unsubstituted heteroaryl having up to 20, such as up to 12, carbon atoms, and 1 to 6, such as 1 to 4, heteroatoms selected from O, S and N, substituted or unsubstituted cycloalkyl or cycloalkenyl having up to 20, such as up to 12, carbon atoms, and substituted or unsubstituted heterocycloalkyl or heterocycloalkenyl having up to 20, such as up to 12, carbon atoms, and 1 to 6, such as 1 to 4, heteroatoms selected from O, S and N,
with the proviso that at least one of R, R¹ and R² is not H and the functional group —O—CR═CR¹R² is derived from an odorant ketone or odorant aldehyde of formula R—C(O)—CHR¹R²; and R³, R⁴ and R⁵ are independently selected from straight-chain or branched, saturated or unsaturated, substituted or unsubstituted hydrocarbon functional groups having 1 to 20 carbon atoms, such as linear or branched alkyl, alkenyl or alkynyl having up to 20, such as up to 12, carbon atoms, substituted or unsubstituted, linear or branched heteroalkyl, heteroalkenyl or heteroalkynyl having up to 20, such as up to 12, carbon atoms, and 1 to 6, such as 1 to 4, heteroatoms selected from O, S and N, substituted or unsubstituted aryl having up to 20, such as up to 12, carbon atoms, substituted or unsubstituted heteroaryl having up to 20, preferably up to 12, carbon atoms, and 1 to 6, such as 1 to 4, heteroatoms selected from O, S and N, cycloalkyl or cycloalkenyl having up to 20, such as up to 12, carbon atoms, and heterocycloalkyl or heterocycloalkenyl having up to 20, such as up to 12, carbon atoms, and 1 to 6, such as 1 to 4, heteroatoms selected from O, S and N.

The aforementioned compounds can be prepared by means of the synthesis routes described in the examples.

In a further aspect, the silyl enol ethers as described herein may be used as a fragrance in liquid or solid washing and cleaning agents or in cosmetic agents, in particular those for treating skin or hair, optionally together with other fragrances, in insect repellents or air care agents in order to extend the fragrance effect of other fragrances.

Yet another aspect is directed to agents containing the silyl enol ethers described herein, in particular washing or cleaning agent, cosmetic agents, air care agents or insect repellents.

Lastly, a method for the long-lasting fragrancing of surfaces, in which a compound as described herein may be applied to the surface to be fragranced, for example (textile) laundry, and said surface is subsequently exposed to conditions which lead to the fragrance being released.

### DETAILED DESCRIPTION

“At least one,” as used herein, refers to 1 or more, for example 2, 3, 4, 5, 6, 7, 8, 9 or more. In connection with components of the compound described herein, this information does not refer to the absolute amount of molecules, but to the type of the component. “At least one compound of formula X” therefore means, for example, one or more different compounds of formula X, i.e., one or more different types of compounds of formula X. Together with stated quantities, the stated amounts refer to the total amount of the correspondingly designated type of constituent, as defined above.

Unless otherwise indicated, all amounts indicated in connection with the agents described herein refer to wt. %, in each case based on the total weight of the agent. Moreover, quantities that relate to at least one component always relate to the total amount of this type of component contained in the agent, unless explicitly indicated otherwise. This means that specified amounts of this type, for example in connection with “at least one fragrance,” refer to the total amount of fragrance contained in the agent.

The term “odorant ketones” is understood to mean fragrances which have a keto group which exhibits keto-enol tautomerism, irrespective of how the molecule is further structured. The same applies to “odorant aldehydes,” which is understood here to mean fragrances which have an aldehyde group which exhibits keto-enol tautomerism, irrespective of how the molecule is further structured. As a prerequisite for the phenomenon of keto-enol tautomerism, it is necessary that the corresponding ketones and aldehydes can be deprotonated in the alpha position or, in alpha beta unsaturated molecules, in the gamma position, i.e. at the alpha or gamma C atom at least one H atom is bonded. Deprotonatable ketones and aldehydes of this kind are therefore the odorant ketones or aldehydes which form the silyl enol ethers. The terms “odorant” and “fragrance” are used interchangeably herein and refer in particular to substances that have a scent that is perceived to be pleasant by humans. In various embodiments, fragrances are substances which are sufficiently volatile to be perceived as odorous by humans as a result of bonding to the olfactory receptor, and the odor of which is perceived to be pleasant. The fragrances or odorants are above all those which are suitable for use in cosmetic, cleaning agent or washing agent compositions. Generally, the fragrances or odorants are liquid at ambient temperatures.

In various embodiments, the odorant aldehyde may be selected from adoxal (2,6,10-trimethyl-9-undecenal), cymene (3-(4-isopropylphenyl)-2-methylpropanal), Florhydral (3-(3-isopropylphenyl)butanal), Helional (3-(3,4-methylenedioxyphenyl)-2-methylpropanal), hydroxycitronellal, lauraldehyde, Lyral (3- and 4-(4-hydroxy-4-methylpentyl)-3-cyclohexene-1-carboxaldehyde), methylnonylacetaldehyde, Lilial (3-(4-tert-butylphenyl)-2-methylpropanal), phenylacetaldehyde, undecylenealdehyde, 2,6,10-trimethyl-9-undecenal, 3-dodecen-1-al, melonal (2,6-dimethyl-5-heptenal), 2,4-dimethyl-3-cyclohexene-1-carboxaldehyde (Triplal), 3-(4-tert-butylphenyl)-propanal, 2-methyl-3-(para-methoxyphenyl)propanal, 2-methyl-4-(2,6,6-trimethyl-2(1)-cyclohexen-1-yl)butanal, cis-/trans-3,7-dimethyl-2,6-octadien-1-al, 3,7-dimethyl-6-octen-1-al, [(3,7-dimethyl-6-octenyl)oxy]acetaldehyde, 1,2,3,4,5,6,7,8-octahydro-8,8-dimethyl-2-naphthaldehyde, 2,4-dimethyl-3-cyclohexene-1-carboxaldehyde, 2-methyl-3-(isopropylphenyl)propanal, 1-decanal, 2,6-dimethyl-5-heptenal, 4-(tricyclo[5.2.1.0(2,6)]-decylidene-8)-butanal, octahydro-4,7-methane-1H-indenecarboxaldehyde, alpha-methyl-3,4-(methylenedioxy)-hydrocinnamaldehyde, m-cymene-7-carboxaldehyde, alpha-methylphenylacetaldehyde, 7-hydroxy-3,7-dimethyloctanal, undecenal, 2,4,6-trimethyl-3-cyclohexene-1-carboxaldehyde, 4-(3)(4-methyl-3-pentenyl)-3-cyclohexenecarboxaldehyde, 1-dodecanal, 2,4-dimethylcyclohexene-3-carboxaldehyde, 4-(4-hydroxy-4-methylpentyl)-3-cyclohexene-1-carboxaldehyde, 7-methoxy-3,7-dimethyloctan-1-al, 2-methylundecanal, 2-methyldecanal, 1-nonanal, 1-octanal, 2,6,10-trimethyl-5,9-undecadienal, 2-methyl-3-(4-tert-butyl)propanal, dihydrocinnamaldehyde, 1-methyl-4-(4-methyl-3-pentenyl)-3-cyclohexene-1-carboxaldehyde, 5- or 6-methoxyhexahydro-4,7-methanindan-1- or -2-carboxaldehyde, 3,7-dimethyloctan-1-al, 1-undecanal, 10-undecen-1-al, 1-methyl-3-(4-methylpentyl)-3-cyclohexenecarboxaldehyde, 7-hydroxy-3,7-dimethyl-octanal, trans-4-decenal, 2,6-nonadienal, para-tolylacetaldehyde, 4-methylphenylacetaldehyde, 2-methyl-4-(2,6,6-trimethyl-1-cyclohexen-1-yl)-2-butenal, 3,5,6-trimethyl-3-cyclohexenecarboxaldehyde, 3,7-dimethyl-2-methylene-6-octenal, phenoxyacetaldehyde, 5,9-dimethyl-4,8-decadienal, peonyaldehyde (6,10-dimethyl-3-oxa-5,9-undecadien-1-al), hexahydro-4,7-methanindan-1-carboxaldehyde, 2-methyloctanal, alpha-methyl-4-(1-methylethyl)benzeneacetaldehyde, 6,6-dimethyl-2-norpinen-2-propionaldehyde, para-methylphenoxyacetaldehyde, 3,5,5-trimethylhexanal, hexahydro-8,8-dimethyl-2-naphthaldehyde, 3-propylbicyclo[2.2.1]-hept-5-en-2-carbaldehyde, 9-decenal, 3-methyl-5-phenyl-1-pentanal, methylnonylacetaldehyde, hexanal, trans-2-hexenal and mixtures thereof.

Non-limiting aldehydes include, without limitation, Lilial, Helional, cyclamenaldehyde, Triplal, melonal, methylundecanal, undecanal, nonanal and octanal.

Suitable ketones include, but are not limited to, 2-undecanone (methylnonyl ketone), methyl-beta-naphthyl ketone, musk indanone (1,2,3,5,6,7-hexahydro-1,1,2,3,3-pentamethyl-4H-inden-4-one), tonalide (6-acetyl-1,1,2,4,4,7-hexamethyltetralin), alpha-damascone, beta-damascone, delta-damascone, iso-damascone, damascenone, methyldihydrojasmonate, menthone, carvone, camphor, Koavone (3,4,5,6,6-pentamethylhept-3-en-2-one), fenchone, alpha-ionone, beta-ionone, gamma-methyl-ionone, fleuramone (2-heptylcyclopentanone), dihydrojasmone, cis-jasmone, 1-(1,2,3,4,5,6,7,8-octahydro-2,3,8,8-tetramethyl-2-naphthalenyl)-ethan-1-one and isomers thereof, methyl cedrenyl ketone, acetophenone, methyl acetophenone, para-methoxy acetophenone, methyl beta-naphthyl ketone, benzyl acetone, para-hydroxyphenyl butanone, celery ketone (3-methyl-5-propyl-2-cyclohexenone), 6-isopropyldeca-hydro-2-naphthone, dimethyloctenone, frescomenthe (2-butan-2-ylcyclohexan-1-one), 4-(1-ethoxyvinyl)-3,3,5,5-tetramethylcyclohexanone, methylheptenone, 2-(2-(4-methyl-3-cyclohexen-1-yl)propyl)cyclopentanone, 1-(p-menthene-6(2)yl)-1-propanone, 4-(4-hydroxy-3-methoxyphenyl)-2-butanone, 2-acetyl-3,3-dimethylnorbornane, 6,7-dihydro-1,1,2,3,3-pentamethyl-4(5H)-indanone, 4-damascol, Dulcinyl (4-(1,3-benzodioxo1-5-yl)butan-2-one), hexalone (1-(2,6,6-trimethyl-2-cyclohexen-1-yl)-1,6-heptadien-3-one), isocyclemone E (2-acetonaphthone-1,2,3,4,5,6,7,8-octahydro-2,3,8,8-tetramethyl), methylcyclocitrone (1-(3,5,6-trimethyl-1-cyclohex-3-enyl)ethanone), methyl lavender ketone (3-hydroxymethylnonan-2-one), Orivone (4-tert-amylcyclohexanone), 4-tert-butylcyclohexanone, delphone (2-pentyl-cyclopentanone), muscone (CAS 541-91-3), neobutenone (1-(5,5-dimethyl-1-cyclo-hexenyl)pent-4-en-1-one), plicatone (CAS 41724-19-0), veloutone (2,2,5-trimethyl-5-pentylcyclopentan-1-one), 2,4,4,7-tetramethyl-oct-6-en-3-one, tetramerane (6,10-dimethylundecen-2-one) and mixtures thereof.

Moreover, in principle all conventional odorant aldehydes and/or odorant ketones which are used in particular for bringing about a smell that is pleasant to humans and are capable of keto-enol tautomerism can be used as odorant aldehydes and/or odorant ketones. Odorant aldehydes and/or odorant ketones of this kind are known to a person skilled in the art and are also described in the patent literature, for example in US 2003/0158079 A1, paragraphs [0154] and [0155]. For further suitable odorants, reference is made to Steffen Arctander, Aroma Chemicals Volume 1 and Volume 2 (published in 1960 and 1969, reprint in 2000; ISBN: 0-931710-37-5 and 0-931710-38-3).

In various embodiments, the silyl enol ethers are those resulting from odorant ketones, in particular those mentioned above. In various embodiments, the odorant ketones are those in which neither the alpha carbon atom nor the beta carbon atom (in each case relative to the oxygen atom) is part of a cyclic group.

In various embodiments, R is a straight-chain or branched, saturated or unsaturated, substituted or unsubstituted hydrocarbon functional group having 1 to 20 carbon atoms and optionally up to 6 heteroatoms, such as a linear or branched alkyl, alkenyl or alkynyl functional group having up to 20, such as up to 12, carbon atoms, i.e. methyl, ethyl, propyl, butyl, pentyl, hexyl, heptyl, octyl, nonyl or decyl.

In various embodiments, R¹ or R² is H and the other functional group is a straight-chain or branched, saturated or unsaturated, substituted or unsubstituted hydrocarbon functional group having 1 to 20 carbon atoms and optionally up to 6 heteroatoms, such as a linear or branched alkyl, alkenyl or alkynyl functional group having up to 20, such as up to 12, carbon atoms. In various embodiments, R¹ and R² may also be H.

When R and R¹ combine with one another in order to form a cyclic group, this cyclic group is selected from substituted or unsubstituted aryl having up to 20, such as up to 12, carbon atoms, substituted or unsubstituted heteroaryl having up to 20, such as up to 12, carbon atoms, and 1 to 6, such as 1 to 4, heteroatoms selected from O, S and N, substituted or unsubstituted cycloalkyl or cycloalkenyl having up to 20, such as up to 12, carbon atoms, and substituted or unsubstituted heterocycloalkyl or heterocycloalkenyl having up to 20, such as up to 12, carbon atoms, and 1 to 6, such as 1 to 4, heteroatoms selected from O, S and N, such as cycloalkyl or cycloalkenyl as defined above.

Generally, in various embodiments, R, R¹ and R² may be selected such that they form, together with the two carbon atoms to which they are bound, an organic functional group having at least 6 carbon atoms.

In various embodiments, R¹ and R² are H and R is a linear, optionally substituted, alkyl functional group having up to 12 carbon atoms. When substituted, the substituent is a cyclic group, for example an aryl or heteroaryl ring, a cycloalkyl or heterocycloalkyl functional group, such as having 5-6 carbon atoms.

“Substituted,” as used herein, means that one or more hydrogen atom(s) is/are replaced in the corresponding functional group by another group, such as selected from hydroxyl, carboxyl, amino, halogen, (hetero)alkyl, (hetero)alkenyl, (hetero)alkynyl, (hetero)aryl, (hetero)cycloalkyl, and (hetero)cycloalkenyl, with the proviso that a given group cannot be substituted with a similar group (i.e. for example, alkyl with alkyl), such as alkylaryl or arylalkyl groups.

“Functional groups” of the above-mentioned odorant ketones and aldehydes are the corresponding enols in which the hydroxyl group is replaced by the silyl ether group having the formula given above, i.e. —O—SiR³R⁴R⁵.

The silicon compounds which are used are completely substituted, i.e. all the functional groups R³, R⁴ and R⁵ are not hydrogen. All of the functional groups R³, R⁴ and R⁵ may be C₁₋₆ hydrocarbons, in particular C₁₋₆ alkyl, such as methyl or ethyl.

The silyl enol ethers are characterized by good hydrolytic stability and can also be used in aqueous media or in preparation processes for granules without suffering excessive loss of activity. In this way, liquid washing and cleaning agents such as liquid detergents, fabric softeners, hand dishwashing detergents, cleaning agents for hard surfaces, floor wiping agents, etc. are also conceivable, as are solid washing and cleaning agents, for example textile washing agent granules, automatic dishwasher detergents or cleaning and scouring agents. The silyl enol ethers can also be used in cosmetic agents for treating skin and hair. Liquid agents such as shower gels, deodorants and hair shampoo are also intended in this case, as well as solid agents such as bars of soap.

As a result of the outstanding suitability of the compounds for use in washing and cleaning agents, silyl enol ethers may be used as described above as a fragrance in liquid or solid washing and cleaning agents and in cosmetic agents, in particular those for treating skin and hair, and in air care agents and insect repellents.

Depending on the nature and intended use of the agents to be fragranced, the silyl enol ethers can be introduced in varying amounts. The silyl enol ethers are usually used in washing and cleaning agents in amounts of from 0.001 to 5 wt. %, such as from 0.01 to 2 wt. %, in each case based on the relevant agent. The agents may in this case contain a silyl enol ether or a plurality of different silyl enol ethers as described herein, the above amounts being based on the total amount of all silyl enol ethers. In insect repellents, the amounts used can be significantly higher; for example, concentrations of 0.001 to 100 wt. %, such as 1 to 50 wt. %, in each case based on the agent, can be used here.

The silyl enol ethers can be used as the sole fragrance, but it is also possible to use fragrance mixtures which consist only in part of the silyl enol ethers. Mixtures of this kind are advantageous in that the components of the fragrance mixture, which are not present as silyl enol ethers of odorant ketones or aldehydes, can also be improved in terms of the durability of the fragrance impression. Thus, fragrance mixtures can be used in particular which contain 1 to 50 wt. %, such as 5 to 40 and in particular at most 30 wt. %, of silyl enol ether based on the fragrance mixture. In other embodiments, in which in particular the delayed fragrance effect of the silyl enol ethers is intended to be used, in the use, advantageously at least 30 wt. %, such as at least 40 wt. % and in particular at least 50 wt. %, of the total perfume contained in the agent is introduced such as the agent via the silyl enol ethers, while the remaining 70 wt. %, such as 60 wt. % and in particular 50 wt. %, of the total perfume contained in the agent is sprayed on in a conventional manner or otherwise introduced into the agent. The use can therefore advantageously be characterized in that the silyl enol ethers are used together with other fragrances.

By dividing the total perfume content of the agent into perfume which is contained in the silyl enol ethers and perfume which has been incorporated conventionally, a variety of product characteristics can be achieved which are only possible by means of the use. Thus, for example, it is conceivable and possible to divide the total perfume content of the agent into two portions x and y, the portion x consisting of adherent, i.e. less volatile, perfume oils and the portion y consisting of more volatile perfume oils.

It is now possible, for example, to prepare washing or cleaning agents in which the portion of the perfume that is introduced into the agent via the silyl enol ethers is mainly composed of adherent odorants. In this way, adherent odorants intended to fragrance the treated articles, in particular textiles, can be “retained” in the product and their effect can develop primarily on the treated laundry as a result. In contrast, the more volatile odorants contribute to a more intensive fragrancing of the agents per se. In this way, it is also possible to prepare washing and cleaning agents which, as an agent, have an odor which differs from the odor of the treated articles. There are hardly any limits to the creativity of perfumers, since the choice of odorants and the choice of method of incorporation into the agent offer virtually limitless possibilities for fragrancing the agents and the articles treated therewith by means of the agents.

Of course, the principle described above can also be reversed by the more volatile odorants being incorporated into the silyl enol ethers and the less volatile, adherent odorants being sprayed on or otherwise incorporated into the agents. In this way, the loss of the more volatile odorants from the packaging during storage and transport is minimized, while the fragrance characteristic of the agents is determined by the more adherent perfumes.

The only limit of this procedure is that the fragrances which are intended to be introduced via the silyl enol ethers originate from the group of odorant ketones and/or aldehydes. The fragrances incorporated into the agents in a conventional manner are not subject to any restrictions. Individual odorant compounds, such as the synthetic products of the ester, ether, aldehyde, ketone, alcohol, and hydrocarbon types, can be used as a perfume oil or fragrance. Odorant compounds of the ester type are, for example, benzyl acetate, phenoxyethyl isobutyrate, p-tert-butylcyclohexyl acetate, linalyl acetate, dimethylbenzylcarbinyl acetate (DMBCA), phenylethyl acetate, benzyl acetate, ethylmethylphenyl glycinate, allylcyclohexyl propionate, styrallyl propionate, benzyl salicylate, cyclohexyl salicylate, Floramate, Melusate, and Jasmacyclate. The ethers include, for example, benzyl ethyl ether and ambroxan; the aldehydes include, for example, the linear alkanals having 8 to 18 C atoms, citral, citronellal, citronellyloxyacetaldehyde, cyclamenaldehyde, Lilial, and bourgeonal; the ketones include, for example, the ionones, α-isomethylionone, and methyl cedryl ketone; the alcohols include anethole, citronellol, eugenol, geraniol, linalool, phenylethyl alcohol, and terpineol; and the hydrocarbons include principally the terpenes such as limonene and pinene. However, mixtures of different odorants are used which together produce an appealing fragrance note.

Perfume oils of this kind can also contain natural odorant mixtures, as are obtainable from plant sources, e.g. pine, citrus, jasmine, patchouli, rose or ylang-ylang oil. Clary sage oil, chamomile oil, clove oil, melissa oil, mint oil, cinnamon leaf oil, lime blossom oil, juniper berry oil, vetiver oil, olibanum oil, galbanum oil, labdanum oil, orange blossom oil, neroli oil, orange peel oil and sandalwood oil are also suitable.

The general description of perfumes that can be used (see above) generally represents the different substance classes of odorants. In order to be perceptible, an odorant must be volatile, wherein, in addition to the nature of the functional groups and the structure of the chemical compound, the molar mass also plays an important role. Therefore, most odorants have molar masses of up to approximately 200 daltons, whereas molar masses of 300 daltons and above represent something of an exception. Due to the differing volatility of odorants, the odor of a perfume or fragrance composed of multiple odorants varies over the course of vaporization, wherein the odor impressions are divided into “top note,” “middle note or body” and “end note or dry out.” Because the perception of an odor also depends to a large extent on the odor intensity, the top note of a perfume or fragrance does not only consist of highly volatile compounds, whereas the end note consists for the most part of less volatile, i.e. adherent, odorants. When composing perfumes, more volatile odorants can be bound for example to specific fixatives, thereby preventing them from evaporating too quickly. The above-described embodiment in which the more volatile odorants and fragrances are present bound in the silyl enol ethers is one such odorant-fixing method. The following subdivision of odorants into “more volatile” and “adherent” odorants therefore provides no information with regard to the odor impression, and as to whether the corresponding odorant is perceived as a top or middle note.

Examples of adherent odorants that can be used within the scope are essential oils such as angelica root oil, aniseed oil, arnica blossom oil, basil oil, bay oil, champaca blossom oil, abies alba oil, abies alba cone oil, elemi oil, eucalyptus oil, fennel oil, spruce needle oil, galbanum oil, geranium oil, ginger grass oil, guaiac wood oil, gurjun balsam oil, helichrysum oil, ho oil, ginger oil, iris oil, cajeput oil, calamus oil, chamomile oil, camphor oil, cananga oil, cardamom oil, cassia oil, pine needle oil, copaiba balsam oil, coriander oil, spearmint oil, caraway oil, cumin oil, lavender oil, lemon grass oil, lime oil, mandarin oil, melissa oil, musk seed oil, myrrh oil, clove oil, neroli oil, niaouli oil, olibanum oil, oregano oil, palmarosa oil, patchouli oil, balsam Peru oil, petitgrain oil, pepper oil, peppermint oil, allspice oil, pine oil, rose oil, rosemary oil, sandalwood oil, celery oil, spike lavender oil, star anise oil, turpentine oil, thuja oil, thyme oil, verbena oil, vetiver oil, juniper berry oil, wormwood oil, wintergreen oil, ylang-ylang oil, hyssop oil, cinnamon oil, cinnamon leaf oil, citronella oil, lemon oil, and cypress oil. However, higher-boiling and solid odorants of natural or synthetic origin may also be used within the scope as adherent odorants or odorant mixtures, i.e. fragrances. These compounds include the compounds indicated in the following and mixtures thereof: ambrettolide, Ambroxan, α-amylcinnamaldehyde, anethole, anisaldehyde, anise alcohol, anisole, anthranilic acid methyl ester, acetophenone, benzylacetone, benzaldehyde, benzoic acid ethyl ester, benzophenone, benzyl alcohol, benzyl acetate, benzyl benzoate, benzyl formate, benzyl valerianate, borneol, bornyl acetate, boisambrene forte, α-bromostyrene, n-decyl aldehyde, n-dodecyl aldehyde, eugenol, eugenol methyl ether, eucalyptol, farnesol, fenchone, fenchyl acetate, geranyl acetate, geranyl formate, heliotropin, heptyne carboxylic acid methyl ester, heptaldehyde, hydroquinone dimethyl ether, hydroxycinnamaldehyde, hydroxycinnamyl alcohol, indole, irone, isoeugenol, isoeugenol methyl ether, isosafrole, jasmone, camphor, carvacrol, carvone, p-cresol methyl ether, coumarin, p-methoxyacetophenone, methyl n-amyl ketone, methylanthranilic acid methyl ester, p-methylacetophenone, methyl chavicol, p-methylquinoline, methyl-β-naphthyl ketone, methyl n-nonyl acetaldehyde, methyl n-nonyl ketone, muscone, β-naphthol ethyl ether, β-naphthol methyl ether, nerol, n-nonyl aldehyde, nonyl alcohol, n-octylaldehyde, p-oxyacetophenone, pentadecanolide, β-phenethyl alcohol, phenylacetaldehyde dimethyl acetal, phenylacetic acid, pulegone, safrole, salicylic acid isoamyl ester, salicylic acid methyl ester, salicylic acid hexyl ester, salicylic acid cyclohexyl ester, santalol, sandelice, skatole, terpineol, thymene, thymol, troenan, γ-undecalactone, vanillin, veratraldehyde, cinnamaldehyde, cinnamyl alcohol, cinnamic acid, cinnamic acid ethyl ester, cinnamic acid benzyl ester.

More volatile odorants include in particular lower-boiling odorants of natural or synthetic origin, which may be used alone or in mixtures. Examples of more volatile odorants are diphenyl oxide, limonene, linalool, linalyl acetate and propionate, melusate, menthol, menthone, methyl-n-heptenone, pinene, phenylacetaldehyde, terpinyl acetate, citral and citronellal.

In addition to the described fragrances, the agents, such washing and cleaning agents, can, of course, contain customary ingredients of agents of this kind. In washing and cleaning agents, primarily surfactants, builder substances, bleaching agents, enzymes, and other active substances should be mentioned in this regard. The essential ingredients of washing and cleaning agents include in particular surfactants.

Depending on the intended purpose of the agents, the surfactant content will be selected so as to be higher or lower. Usually, the surfactant content of washing agents is between 10 and 40 wt. %, such as between 12.5 and 30 wt. %, and in particular between 15 and 25 wt. %, while cleaning agents for automatic dishwashing contain between 0.1 and 10 wt. %, such as between 0.5 and 7.5 wt. %, and in particular between 1 and 5 wt. % of surfactants.

These surface-active substances come from the group of anionic, non-ionic, zwitterionic or cationic surfactants, anionic and non-ionic surfactants being are better for use when considering economical reasons and the performance spectrum thereof during washing and cleaning.

Anionic surfactants that are used are those of the sulfonate and sulfate types, for example. Surfactants of the sulfonate type that can be used are in this case C₉₋₁₃ alkylbenzene sulfonates, olefin sulfonates, i.e. mixtures of alkene and hydroxyalkane sulfonates, and disulfonates, as they are obtained, for example, from C₁₂₋₁₈ monoolefins having a terminal or internal double bond by way of sulfonation with gaseous sulfur trioxide and subsequent alkaline or acid hydrolysis of the sulfonation products. Alkane sulfonates obtained from C₁₂₋₁₈ alkanes, for example by way of sulfochlorination or sulfoxidation with subsequent hydrolysis or neutralization, are also suitable. Likewise, the esters of α-sulfofatty acids (ester sulfonates) are suitable, for example the α-sulfonated methyl esters of hydrogenated coconut fatty acids, palm kernel fatty acids or tallow fatty acids.

Sulfated fatty acid glycerol esters are further suitable anionic surfactants. Fatty acid glycerol esters are understood to mean the monoesters, diesters and triesters and the mixtures thereof, as they are obtained during production by way of esterification of a monoglycerol having 1 to 3 mol of fatty acid or during the transesterification of triglycerides having 0.3 to 2 mol of glycerol. Non-limiting sulfated fatty acid glycerol esters are in this case the sulfation products of saturated fatty acids having 6 to 22 carbon atoms, for example of caproic acid, caprylic acid, capric acid, myristic acid, lauric acid, palmitic acid, stearic acid or behenic acid.

The alkali salts and in particular the sodium salts of the sulfuric acid half-esters of C₁₂-C₁₈ fatty alcohols, for example from coconut fatty alcohol, tallow fatty alcohol, lauryl alcohol, myristyl alcohol, cetyl alcohol or stearyl alcohol, or of C₁₀-C₂₀ oxo alcohols and the half-esters of secondary alcohols having these chain lengths may be or include alk(en)yl sulfates. Alk(en)yl sulfates of the mentioned chain length that contain a synthetic straight-chain alkyl functional group prepared on a petrochemical basis and have a degradation behavior similar to that of the adequate compounds based on fatty chemical raw materials are also possible. From a washing perspective, C₁₂-C₁₆ alkyl sulfates, C₁₂-C₁₅ alkyl sulfates and C₁₄-C₁₅ alkyl sulfates may be used.

The sulfuric acid monoesters of straight-chain or branched C₇-C₂₁ alcohols ethoxylated with 1 to 6 mol of ethylene oxide, such as 2-methyl-branched C₉₋₁₁ alcohols having, on average, 3.5 mol ethylene oxide (EO) or C₁₂₋₁₈ fatty alcohols having 1 to 4 EO, are also suitable. Due to the high foaming behavior thereof, they are used in cleaning agents only in relatively small amounts, for example in amounts of from 1 to 5 wt. %.

Further suitable anionic surfactants are also the salts of alkyl sulfosuccinic acid, which are also referred to as sulfosuccinates or as sulfosuccinic acid esters and represent the monoesters and/or diesters of sulfosuccinic acid with alcohols, such as fatty alcohols, and in particular ethoxylated fatty alcohols. Non-limiting sulfosuccinates contain C₈-C₁₈ fatty alcohol functional groups or mixtures thereof. In particular, sulfosuccinates contain a fatty alcohol functional group that is derived from ethoxylated fatty alcohols, which, considered in isolation, represent non-ionic surfactants (for description see below). In this case, sulfosuccinates of which the fatty alcohol functional groups derive from ethoxylated fatty alcohols exhibiting a restricted homolog distribution may be used. Likewise, it is also possible to use alk(en)yl succinic acid having such as 8 to 18 carbon atoms in the alk(en)yl chain, or the salts thereof.

Further anionic surfactants that can also be used are in particular soaps. Saturated fatty acid soaps are suitable, such as the salts of lauric acid, myristic acid, palmitic acid, stearic acid, hydrogenated erucic acid and behenic acid, and in particular soap mixtures derived from natural fatty acids, such as coconut fatty acids, palm kernel fatty acids or tallow fatty acids.

The anionic surfactants, including the soaps, can be present in the form of the sodium, potassium or ammonium salts thereof, or as soluble salts of organic bases, such as monoethanolamine, diethanolamine or triethanolamine. The anionic surfactants are present in the form of the sodium, potassium or magnesium salts thereof, and in particular in the form of the sodium salts.

There are no general conditions that must be adhered to that would stand in the way of having a degree of freedom in terms of the formulation when selecting the anionic surfactants. However, non-limiting agents have a soap content that exceeds 0.2 wt. %, based on the total weight of the washing and cleaning agent prepared in step d). Anionic surfactants which are to be used are in this case alkylbenzene sulfonates and fatty alcohol sulphates, non-limiting washing agent shaped bodies containing 2 to 20 wt. %, such as 2.5 to 15 wt. %, and in particular 5 to 10 wt. %, of fatty alcohol sulfate(s), in each case based on the weight of the agents.

Non-ionic surfactants that are used are alkoxylated, advantageously ethoxylated, in particular primary alcohols having 8 to 18 C atoms and, on average, 1 to 12 mols of ethylene oxide (EO) per mol of alcohol, in which the alcohol functional group can be linear or methyl-branched in the 2 position, or can contain linear and methyl-branched functional groups in admixture, as are usually present in oxo alcohol functional groups. However, alcohol ethoxylates having linear functional groups of alcohols of native origin having 12 to 18 C atoms, for example of coconut alcohol, palm alcohol, tallow fatty alcohol or oleyl alcohol, and an average of 2 to 8 EO per mol of alcohol, may be used. Non-limiting ethoxylated alcohols include, for example, C₁₂₋₁₄ alcohols having 3 EO or 4 EO, C₉₋₁₁ alcohols having 7 EO, C₁₃₋₁₅ alcohols having 3 EO, 5 EO, 7 EO or 8 EO, C₁₂₋₁₈ alcohols having 3 EO, 5 EO or 7 EO, and mixtures thereof, such as mixtures of C₁₂₋₁₄ alcohol having 3 EO and C₁₂₋₁₈ alcohol having 5 EO. The degrees of ethoxylation indicated represent statistical averages that can correspond to an integer or a fractional number for a specific product. Non-limiting alcohol ethoxylates have a narrowed homolog distribution (narrow range ethoxylates, NRE). In addition to these non-ionic surfactants, fatty alcohols having more than 12 EO can also be used. Examples of these are tallow fatty alcohols having 14 EO, 25 EO, 30 EO, or 40 EO.

Another class of non-ionic surfactants that are used, which are used either as the sole non-ionic surfactant or in combination with other non-ionic surfactants, is alkoxylated, such as ethoxylated or ethoxylated and propoxylated fatty acid alkyl esters, such as having 1 to 4 carbon atoms in the alkyl chain, in particular fatty acid methyl esters, such as those described for example in the Japanese patent application JP 58/217598 or those prepared according to the method described in the international patent application WO-A-90/13533.

Another class of non-ionic surfactants that can advantageously be used is the alkyl polyglycosides (APG). Alkyl polyglycosides that can be used have the general formula RO(G)_{z}, in which R represents a linear or branched, in particular methyl-branched at the 2-position, saturated or unsaturated aliphatic functional group having 8 to 22, such as 12 to 18, C atoms, and G is the symbol that represents a glycose unit having 5 or 6 C atoms, such as glucose. The degree of glycosidation z is in this case between 1.0 and 4.0, such as between 1.0 and 2.0, and in particular between 1.1 and 1.4. Linear alkyl polyglycosides are used, i.e. alkyl polyglycosides in which the polyglycol functional group is a glucose functional group and the alkyl functional group is an n-alkyl functional group.

Non-ionic surfactants of the amine oxide type, for example N-cocoalkyl-N,N-dimethylamine oxide and N-tallowalkyl-N,N-dihydroxyethylamine oxide, and of the fatty acid alkanolamide type may also be suitable. The quantity of these non-ionic surfactants is no more than that of the ethoxylated fatty alcohols, in particular no more than half thereof.

Further suitable surfactants are polyhydroxy fatty acid amides of formula (III),

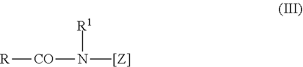

in which RCO represents an aliphatic acyl functional group having 6 to 22 carbon atoms, R¹ represents hydrogen, an alkyl functional group or hydroxyalkyl functional group having 1 to 4 carbon atoms, and [Z] represents a linear or branched polyhydroxyalkyl functional group having 3 to 10 carbon atoms and 3 to 10 hydroxyl groups. The polyhydroxy fatty acid amides are known substances that can usually be obtained by the reductive amination of a reducing sugar with ammonia, an alkylamine or an alkanolamine, and subsequent acylation with a fatty acid, a fatty acid alkyl ester or a fatty acid chloride.

The group of polyhydroxy fatty acid amides also includes compounds of formula (IV),

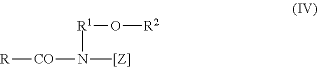

in which R represents a linear or branched alkyl or alkenyl functional group having 7 to 12 carbon atoms, R¹ represents a linear, branched or cyclic alkyl functional group or an aryl functional group having 2 to 8 carbon atoms, and R² represents a linear, branched or cyclic alkyl functional group or an aryl functional group or an oxy alkyl functional group having 1 to 8 carbon atoms, C₁₋₄ alkyl or phenyl functional groups may be used, and [Z] represents a linear polyhydroxy alkyl functional group, the alkyl chain of which is substituted with at least two hydroxyl groups, or alkoxylated, such as ethoxylated or propoxylated derivatives of this functional group. [Z] is obtained by the reductive amination of a reduced sugar, for example glucose, fructose, maltose, lactose, galactose, mannose or xylose. The N-alkoxy-substituted or N-aryloxy-substituted compounds can be converted, in the presence of an alkoxide as the catalyst, into the desired polyhydroxy fatty acid amides by reacting these with fatty acid methyl esters, for example according to the teaching of the international application WO-A-95/07331.

Builder substances are another significant group of washing and cleaning agent ingredients. This substance class is understood to cover both organic and inorganic builder substances. These are compounds which may carry out a carrier function in the agents and also act as a water softening substance during use.

Usable organic builder substances are, for example, the polycarboxylic acids that can be used in the form of the sodium salts thereof, polycarboxylic acids being understood to mean those carboxylic acids that carry more than one acid function. These include, for example, citric acid, adipic acid, succinic acid, glutaric acid, malic acid, tartaric acid, maleic acid, fumaric acid, saccharic acids, aminocarboxylic acids, nitrilotriacetic acid (NTA), provided that the use thereof is not objectionable for ecological reasons, and mixtures thereof. Non-limiting salts are the salts of polycarboxylic acids such as citric acid, adipic acid, succinic acid, glutaric acid, tartaric acid, saccharic acids, methylglycinediacetic acid, glutamine diacetic acid, and mixtures thereof. The acids can also be used per se. In addition to the builder effect thereof, the acids typically also have the property of being an acidification component and are thus also used, for example in the granules, for setting a lower and milder pH of washing or cleaning agents. Particularly noteworthy here are citric acid, succinic acid, glutaric acid, adipic acid, gluconic acid, methylglycinediacetic acid, glutamine diacetic acid and any mixtures thereof.

Polymeric polycarboxylates are also suitable as builders. These are, for example, the alkali metal salts of polyacrylic acid or polymethacrylic acid, for example those having a relative molecular mass of from 500 to 70,000 g/mol. This substance class has already been described in detail above. The (co)polymeric polycarboxylates may be used either as a powder or an aqueous solution. The content of (co)polymeric polycarboxylates in the agent is 0.5 to 20 wt. %, in particular 3 to 10 wt. %.

To improve water solubility, the polymers can also contain allyl sulfonic acids, such as allyloxybenzene sulfonic acid and methallyl sulfonic acid, as in EP-B-0 727 448 for example, as monomers. Biodegradable polymers composed of more than two different monomer units are also possible, for example those that, according to DE-A-43 00 772, contain salts of acrylic acid and of maleic acid, and vinyl alcohol or vinyl alcohol derivatives as monomers or, according to DE-C-42 21 381, salts of acrylic acid and of 2-alkylallylsulfonic acid and sugar derivatives as monomers. Further copolymers are those that are described in the German patent applications DE-A-43 03 320 and DE-A-44 17 734 and comprise acrolein and acrylic acid/acrylic acid salts or acrolein and vinyl acetate as monomers. Polymeric aminodicarboxylic acids, the salts thereof or the precursors thereof should likewise be mentioned as further builders. Also possible are polyaspartic acids or the salts and derivatives thereof, of which it is disclosed in the German patent application DE-A-195 40 086 that they also exhibit a bleach-stabilizing effect in addition to cobuilder properties.

Additional suitable builders are polyacetals, which may be obtained by reacting dialdehydes with polyolcarboxylic acids which have 5 to 7 C atoms and at least 3 hydroxyl groups, for example as described in the European patent application EP-A-0 280 223. Non-limiting polyacetals are obtained from dialdehydes such as glyoxal, glutaraldehyde, terephthalaldehyde and mixtures thereof, and from polyol carboxylic acids such as gluconic acid and/or glucoheptonic acid.

Further suitable organic builders are dextrins, for example oligomers or polymers of carbohydrates, which can be obtained by the partial hydrolysis of starches. The hydrolysis can be carried out according to customary methods, for example acid- or enzyme-catalyzed methods. These dextrins are hydrolysis products having average molar masses in the range of from 400 to 500,000 g/mol. In this case, a polysaccharide having a dextrose equivalent (DE) in the range of from 0.5 to 40, in particular from 2 to 30, is preferred, DE being a customary measure for the reducing effect of a polysaccharide compared to dextrose, which has a DE of 100. It is possible to use both maltodextrins having a DE between 3 and 20 and dried glycose syrups having a DE between 20 and 37, and what are known as yellow dextrins and white dextrins having higher molar masses in the range of from 2,000 to 30,000 g/mol. A dextrin is described in the British patent application 94 19 091. Oxidized derivatives of dextrins of this type are the reaction products thereof with oxidizing agents which are capable of oxidizing at least one alcohol function of the saccharide ring to form a carboxylic acid function. Oxidized dextrins of this kind and methods for the preparation thereof are known, for example, from the European patent applications EP-A-0 232 202, EP-A-0 427 349, EP-A-0 472 042 and EP-A-0 542 496, and the international patent applications WO 92/18542, WO-A-93/08251, WO-A-93/16110, WO-A-94/28030, WO-A-95/07303, WO-A-95/12619 and WO-A-95/20608. An oxidized oligosaccharide according to the German patent application DE-A-196 00 018 is also suitable. A product that is oxidized on Cs of the saccharide ring can be particularly advantageous.

Oxydisuccinates and other derivatives of disuccinates, such as ethylenediamine disuccinate, are further suitable cobuilders. Ethylenediamine-N,N′-disuccinate (EDDS), the synthesis of which is described in U.S. Pat. No. 3,158,615, for example, is used in the form of the sodium or magnesium salts thereof. Glycerol disuccinates and glycerol trisuccinates, as they are described for example in the US patent specifications U.S. Pat. Nos. 4,524,009, 4,639,325, in the European patent application EP-A-0 150 930 and in the Japanese patent application JP 93/339896, are also further optional in this context. Suitable use amounts are 3 to 15 wt. % in zeolite-containing and/or silicate-containing formulations. Further organic cobuilders that can be used are, for example, acetylated hydroxycarboxylic acids or the salts thereof, which optionally can also be present in lactone form and comprise at least 4 carbon atoms and at least one hydroxy group, as well as no more than two acid groups. Cobuilders of this kind are described, for example, in the international patent application WO-A-95/20029.

A further substance class having cobuilder properties is that of phosphonates. These include, in particular, hydroxyalkane and aminoalkane phosphonates. Among the hydroxyalkane phosphonates, 1-hydroxyethane-1,1-diphosphonate (HEDP) is of particular importance as a cobuilder. It is used as a sodium salt, the disodium salt reacting neutral and the tetrasodium salt reacting alkaline (pH 9). Possible aminoalkane phosphonates include ethylenediamine tetramethylene phosphonate (EDTMP), diethylentriamine pentamethylene phosphonate (DTPMP) and the higher homologues thereof. They are used in the form of the neutrally reacting sodium salt, for example as the hexasodium salt of EDTMP or as the hepta- and octa-sodium salt of DTPMP. From the class of phosphonates, HEDP is used as a builder. The aminoalkane phosphonates additionally have a pronounced heavy-metal-binding power. Accordingly, if the agents also contain bleach, aminoalkane phosphonates, in particular DTPMP, or to use mixtures of the mentioned phosphonates may be used.

Moreover, all compounds that are able to form complexes with alkaline earth ions can be used as cobuilders.

A used inorganic builder is finely crystalline, synthetic and bound water-containing zeolite. The microcrystalline, synthetic and bound water-containing zeolite used is zeolite A and/or zeolite P. Zeolite X and mixtures of A, X and/or P, for example a co-crystallizate from zeolites A and X, are also suitable, however. The zeolite can be used as a spray-dried powder or also as an undried, stabilized suspension that is still moist from production. If zeolite is used in the form of a suspension, it may contain small additional amounts of non-ionic surfactants as stabilizers, for example from 1 to 3 wt. %, based on the zeolite, of ethoxylated C₁₂-C₁₈ fatty alcohols having 2 to 5 ethylene oxide groups, C₁₂-C₁₄ fatty alcohols having 4 to 5 ethylene oxide groups or ethoxylated isotridecanols. Suitable zeolites have an average particle size of less than 10 μm (volume distribution; measuring method: Coulter counter) and contain from 18 to 22 wt. %, and in particular from 20 to 22 wt. %, of bound water. In embodiments, zeolites are contained in the premix in amounts of from 10 to 94.5 wt. %, such as for zeolite to be contained in amounts of from 20 to 70 wt. %, in particular from 30 to 60 wt. %.

Suitable partial substitutes for zeolites are phyllosilicates of natural and synthetic origin. Phyllosilicates of this kind are known from patent applications DE-A-23 34 899, EP-A-0 026 529 and DE-A-35 26 405, for example. The usability thereof is not limited to a specific composition or structural formula. However, in this case smectites may be used or included, in particular bentonites. Crystalline, layered sodium silicates of the general formula NaMSiₓO₂ₓ₊₁.yH₂O, where M is sodium or hydrogen, x is a number from 1.9 to 4 and y is a number from 0 to 20, and non-limiting values for x are 2, 3 or 4, are also suitable as zeolite or phosphate substitutes. Crystalline phyllosilicates of this kind are described, for example, in European patent application EP-A-0 164 514. Non-limiting crystalline phyllosilicates of the aforementioned formula are those in which M represents sodium and x assumes the values 2 or 3. Both β and δ-sodium disilicates Na₂Si₂O₅.yH₂O may be included.

The builder substances also include amorphous sodium silicates having an Na₂O:SiO₂ modulus of 1:2 to 1:3.3, such as of 1:2 to 1:2.8, and in particular of 1:2 to 1:2.6, which are delayed in dissolution and have secondary washing properties. The retarded dissolution compared to conventional amorphous sodium silicates can have been caused in a variety of ways, for example by way of surface treatment, compounding, compacting/compression or over-drying. The term “amorphous” is also understood to mean “X-ray amorphous.” This means that the silicates do not supply any sharp X-ray reflexes in X-ray diffraction experiments, such as those that are typical of crystalline substances, but at best one or more maxima of the scattered X-rays, which have a width of several degree units of the diffraction angle. However, even particularly good builder properties may very well be achieved when the silicate particles supply washed-out or even sharp diffraction maxima in electron diffraction experiments. This should be interpreted such that the products comprise microcrystalline regions measuring 10 to several hundred nm, values up to a maximum of 50 nm, and in particular up to a maximum of 20 nm, may be included. X-ray amorphous silicates of this kind, which likewise exhibit delayed dissolution compared with conventional water glasses, are described in the German patent application DE-A-44 00 024, for example. In particular, compressed/compacted amorphous silicates, compounded amorphous silicates and overdried X-ray amorphous silicates are preferred, in particular the overdried silicates also occurring as carriers in the granules or being used as carriers in the method.

It is self-evidently also possible to use the generally known phosphates as builders, provided that the use thereof should not be avoided for ecological reasons. Sodium salts of orthophosphates, pyrophosphates and in particular tripolyphosphates are particularly suitable. The content thereof is generally no more than 25 wt. %, such as no more than 20 wt. %, in each case based on the finished agent. In embodiments, the agents are phosphate-free, i.e. contain less than 1 wt. % of phosphates of this kind.

In addition to the aforementioned components, the washing and cleaning agents can additionally contain one or more substances from the group of bleaching agents, bleach activators, enzymes, pH adjusting agents, fluorescing agents, dyes, suds suppressors, silicone oils, anti-redeposition agents, optical brighteners, graying inhibitors, dye transfer inhibitors, corrosion inhibitors and silver protecting agents. Suitable agents are known in the prior art.

This list of washing and cleaning agent ingredients is by no means exhaustive, but merely reflects the most essential typical ingredients of agents of this kind. In particular, if the preparations are liquid or gel-like, organic solvents can also be contained in the agents. These are monohydric or polyhydric alcohols having 1 to 4 carbon atoms. Non-limiting alcohols in such agents are ethanol, 1,2-propanediol, glycerol, and mixtures of these alcohols. In embodiments, agents of this type contain 2 to 12 wt. % of alcohols of this kind.

In principle, the agents can be in different physical states. In a non-limiting embodiment, the washing or cleaning agents are liquid or gel-like agents, in particular liquid detergents or liquid dishwashing detergents or cleaning gels, it being possible for these to also in particular be gel-like cleaning agents for flushing toilets. Gel-like cleaning agents of this kind for flushing toilets are described, for example, in the German patent application DE-A-197 158 72.

Further typical cleaning agents that may include the silyl enol ethers are liquid or gel-like cleaners for hard surfaces, in particular those known as all-purpose cleaners, glass cleaners, floor or bathroom cleaners, and special embodiments of such cleaners, which also include acid or alkaline forms of all-purpose cleaners, as well as glass cleaners having what is known as anti-rain action. These liquid cleaning agents can be present either in one or in multiple phases. In an embodiment, the cleaners have two different phases.

In the broadest sense, “cleaner” is a designation for formulations which usually contain surfactants and have a very wide range of use, and, as a result, a widely varying composition. The most important market segments are household cleaners, industrial (technical) and institutional cleaners. Based on the pH value, a distinction is made between alkaline, neutral and acid cleaners, and according to the form in which the product is offered, a distinction is made between liquid and solid cleaners (including in tablet form). Contrary to dishwashing agents, for example, which can likewise be categorized in the cleaner product group, cleaners for hard surfaces exhibit an optimal application profile, both in the concentrated state and in a diluted aqueous solution, in conjunction with mechanical energy. Cold cleaners develop the action thereof without an increased temperature. Above all, surfactants and/or alkali carriers, alternatively acids, optionally also solvents such as glycol ethers and lower alcohols, are decisive for the cleaning effect. In general, the formulations also include builders, and, depending on the type of cleaner, also bleaching agents, enzymes, microbe-mitigating or disinfecting additives, perfume oils and dyes. Cleaners can also be formulated as microemulsions. To a large degree, the cleaning success depends on the type of dirt, which also varies widely geographically, and the properties of the surfaces to be cleaned.

The cleaners can contain anionic, non-anionic, amphoteric or cationic surfactants or surfactant mixtures of one, a plurality or all of these surfactant classes as the surfactant component. The cleaners contain surfactants in amounts, based on the composition, of 0.01 to 30 wt. %, such as 0.1 to 20 wt. %, in particular 1 to 14 wt. %, e.g. 3 to 10 wt. %.

Suitable non-ionic surfactants in all-purpose cleaners of this kind are, for example, C₈-C₁₈ alkyl alcohol polyglycol ethers, alkyl polyglycosides and nitrogen-containing surfactants and mixtures thereof, in particular of the first two. The agents contain non-ionic surfactants in amounts, based on the composition, of 0 to 30 wt. %, such as 0.1 to 20 wt. %, in particular 0.5 to 14 wt. %, and e.g. 1 to 10 wt. %.

Ca₈₋₁₈ alkyl alcohol polypropylene glycol/polyethylene glycol ethers represent known non-ionic surfactants. They can be described by the formula RⁱO—(CH₂CH(CH₃)O)ₚ(CH₂CH₂O)ₑ—H, in which Rⁱ represents a linear or branched aliphatic alkyl and/or alkenyl functional group having 8 to 18 carbon atoms, p represents 0 or numbers from 1 to 3, and e represents numbers from 1 to 20. The C₈₋₁₈ alkyl alcohol polyglycol ethers can be obtained by way of addition of propylene oxide and/or ethylene oxide to alkyl alcohols, e.g. fatty alcohols. Typical examples are polyglycol ethers in which Rⁱ represents an alkyl functional group having 8 to 18 carbon atoms, p represents 0 to 2, and e represents numbers from 2 to 7. Non-limiting representatives are, for example, C₁₀-C₁₄ fatty alcohol+1 PO+6EO ether (p=1, e=6), and C₁₂-C₁₈ fatty alcohol+7EO ether (p=0, e=7) and the mixtures thereof.

It is also possible to use end-capped C₈-C₁₈ alkyl alcohol polyglycol ethers, which is to say compounds in which the free OH group is etherified. The end-capped C₈₋₁₈ alkyl alcohol polyglycol ethers can be obtained according to relevant methods of preparative organic chemistry. C₈₋₁₈ alkyl alcohol polyglycol ethers are reacted, in the presence of bases, with alkyl halides, in particular butyl or benzyl chloride. Typical examples are mixed ethers, in which R′ represents a technical fatty alcohol functional group, such as a C_{12/14} coconut alkyl functional group, p represents 0, and e represents 5 to 10, which mixed ethers are capped with a butyl group.

Non-limiting non-ionic surfactants are also the alkyl polyglycosides described further above. Nitrogen-containing surfactants, such as fatty acid polyhydroxyamides, for example glucamides, and ethoxylates of alkyl amines, vicinal diols and/or carboxylic acid amides that have alkyl groups having 10 to 22 C atoms, such as 12 to 18 C atoms, may be contained as further non-ionic surfactants. The degree of ethoxylation of these compounds is generally between 1 and 20, such as between 3 and 10. Ethanolamide derivatives of alkanoic acids having 8 to 22 C atoms, such as 12 to 16 C atoms, may be included. Particularly suitable compounds include lauric acid, myristic acid and palmitic acid monoethanolamides.

Suitable anionic surfactants for all-purpose cleaners are C₈₋₁₈ alkyl sulfates, C₈₋₁₈ alkyl ether sulfates, i.e. the sulfation products of alcohol ethers and/or C₈₋₁₈ alkylbenzenesulfonates, and C₈₋₁₈ alkanesulfonates, C₈₋₁₈ α-olefinsulfonates, sulfonated C₈₋₁₈ fatty acids, in particular dodecylbenzenesulfonate, C₈₋₂₂ carboxylic acid amide ether sulfates, sulfonosuccinic acid mono- and di-C₁₋₁₂ alkyl esters, C₈₋₁₈ alkyl polyglycol ether carboxylates, C₈₋₁₈ N-acyl taurides, C₈₋₁₈ N-sarcosinates and C₈₋₁₈ alkyl isethionates or mixtures thereof. They are used in the form of the alkali metal and alkaline-earth metal salts thereof, in particular sodium, potassium and magnesium salts, and ammonium- and mono-, di-, tri- or tetra-alkyl ammonium salts, and, in the case of the sulfonates, also in the form of the corresponding acid thereof, such as dodecylbenzene sulfonic acid. The agents contain anionic surfactants in amounts, based on the composition, of 0 to 30 wt. %, such as 0.1 to 20 wt. %, in particular 1 to 14 wt. %, e.g. 2 to 10 wt. %.

Due to the foam-controlling properties thereof, the all-purpose cleaners can also contain soaps, i.e. alkali or ammonium salts of saturated or unsaturated C₆₋₂₂ fatty acids. The soaps may be used in an amount of up to 5 wt. %, such as from 0.1 to 2 wt. %.

Suitable amphoteric surfactants are, for example, betaines of formula (Rⁱⁱ)(Rⁱⁱⁱ)(R^{iv})N⁺CH₂COO⁻, in which Rⁱⁱ represents an alkyl functional group, which is optionally interrupted by heteroatoms or heteroatom groups, having 8 to 25, such as 10 to 21, carbon atoms, and Rⁱⁱⁱ and R^{iv} represent identical or different alkyl functional groups having 1 to 3 carbon atoms, in particular C₁₀₋₁₈ alkyl dimethyl carboxymethyl betaine and C₁₁₋₁₇ alkyl amido propyl dimethyl carboxymethyl betaine. The agents contain amphoteric surfactants in amounts, based on the composition, of 0 to 15 wt. %, such as 0.01 to 10 wt. %, and in particular 0.1 to 5 wt. %.

Suitable cationic surfactants are, inter alia, the quaternary ammonium compounds of formula (R^{v})(R^{vi})(R^{vii})(R^{viii})N⁺X⁺, in which R^{v} to R^{viii} represent four identical or different, in particular two long-chain and two short-chain, alkyl functional groups, and X⁻ represents an anion, in particular a halide ion, for example didecyl dimethyl ammonium chloride, alkyl benzyl didecyl ammonium chloride and the mixtures thereof. The agents contain cationic surfactants in amounts, based on the composition, of 0 to 10 wt. %, such as 0.01 to 5 wt. %, and in particular 0.1 to 3 wt. %.

In an embodiment, the cleaners contain anionic and non-ionic surfactants together, such as C₈₋₁₈ alkylbenzene sulfonates, C₈₋₁₈ alkyl sulfates and/or C₈₋₁₈ alkyl ether sulfates in addition to C₈₋₁₈ alkyl alcohol polyglycol ethers and/or alkyl polyglycosides, in particular C₈₋₁₈ alkylbenzene sulphonates in addition to C₈₋₁₈ alkyl alcohol polyglycol ethers.

The cleaners can also contain builders. Suitable builders are, for example, alkali metal gluconates, citrates, nitrilotriacetates, carbonates and bicarbonates, in particular sodium gluconate, citrate and nitrilotriacetate, and sodium and potassium carbonate and bicarbonate, and alkali metal and alkaline-earth metal hydroxides, in particular sodium and potassium hydroxide, ammonia and amines, in particular monoethanolamine and triethanolamine, and the mixtures thereof. These also include the salts of glutaric acid, succinic acid, adipic acid, tartaric acid and benzene hexacarboxylic acid as well as phosphonates and phosphates. The agents contain builders in amounts, based on the composition, of 0 to 20 wt. %, such as 0.01 to 12 wt. %, in particular 0.1 to 8 wt. %, and e.g. 0.3 to 5 wt. %, the amount of sodium hexametaphospate, excluding the agents used, being limited to 0 to 5 wt. %, however. As electrolytes, the builder salts are phase separation agents at the same time.

In addition to the cited components, the cleaners may contain further auxiliary agents and additives, as are common in such agents. These include in particular polymers, soil release active ingredients, solvents (e.g. ethanol, isopropanol, glycol ether), solubilizers, hydrotropic substances (e.g. cumene sulfonate, octyl sulfate, butyl glucoside, butyl glycol), cleaning boosters, viscosity regulators (e.g. synthetic polymers such as polysaccharides, polyacrylates, naturally occurring polymers and the derivatives thereof such as xanthan gum, other polysaccharides and/or gelatin), pH regulators (e.g. citric acid, alkanolamines or NaOH), disinfectants, antistatic agents, preservatives, bleaching systems, enzymes, dyes, and opacifiers or skin protection agents, as they are described in EP-A-0 522 506. The amount of additives of this type in the cleaning agent is usually no greater than 12 wt. %. The lower limit of what is used depends on the additive type and, for dyes, may be as low as 0.001 wt. % or less, for example. The amount of auxiliary agents is between 0.01 and 7 wt. %, in particular 0.1 and 4 wt. %.

The pH value of the all-purpose cleaners can be varied over a wide range; however, a range from 2.5 to 12, in particular from 5 to 10.5, is possible. The pH value is understood to mean the pH value of the agent in the form of the temporary emulsion.

All-purpose cleaner formulations of this kind can be modified for any purpose. One particular embodiment is the glass cleaners. In cleaners of this kind it is essential that stains or outlines remain. In particular, it is a problem in this case that, after cleaning, water condenses on these surfaces and results in what is known as the fogging effect. It is likewise undesirable when what are known as rain stains remain on glass panes exposed to rain. This effect is known as the rain effect or anti-rain effect. These effects can be prevented by suitable additives in glass cleaners.

In another embodiment, the agents are powdery or granular agents. The agents can in this case have any bulk densities. The spectrum of possible bulk densities ranges from low bulk densities of less than 600 g/l, for example 300 g/l, through the range of average bulk densities from 600 to 750 g/l, to the range of high bulk densities of at least 750 g/l.

Arbitrary methods which are known from the prior art are suitable for preparing agents of this kind.

Cosmetic agents for treating hair or skin, may contain the silyl enol ethers described herein in the amounts already described above in connection with the other agents. In an embodiment, the cosmetic agents are aqueous preparations that contain surface-active ingredients and that are suitable in particular for treating keratin fibers, in particular human hair, or for treating skin.

The mentioned hair treatment agents are in this case in particular agents for treating human scalp hair. The most common agents of this category can be divided into hair washing agents, hair care agents, hair setting and hair styling agents, hair dyes and hair removal agents. The agents which contain surface-active ingredients may include in particular hair washing agents and hair care agents. These aqueous preparations are typically present in a liquid to pasty form.

Fatty alcohol polyglycol ether sulfates (ether sulfates, alkyl ether sulfates), in part in combination with other usually anionic surfactants, are used predominantly for the most important group of ingredients, specifically the surface-active ingredients or washing-active substances. In addition to good cleaning power and insensitivity to water hardness, shampoo surfactants are intended to have good skin and mucosal compatibility. In accordance with statutory provisions, they have to have good biodegradability. In addition to the alkyl ether sulfates, non-limiting agents can additionally contain further surfactants such as alkyl sulfates, alkyl ether carboxylates, such as having degrees of ethoxylation from 4 to 10, and surfactant protein/fatty acid condensates.

Hair shampoos contain perfume oils to produce a pleasant fragrance note. In this case the shampoos may contain only the silyl enol ethers, but the hair shampoos may also contain not only these but also other fragrances. All conventional fragrances permitted in hair shampoos may be used in this case.

The goal of hair care agents is to preserve the natural state of newly regrown hair for as long as possible, and to restore the same if damaged. Features that characterize this natural state are a silky shine, low porosity, a resilient and soft volume, and a pleasantly smooth feel. An important prerequisite for this is a clean, not overly oily scalp that is free of dandruff. Today, hair care agents include a large number of different products, the most important representatives of which are referred to as pre-treatment agents, hair tonics, hairdressing agents, hair rinses and masque products.

The aqueous preparations for treating skin are in particular preparations for human skin care. This care begins with cleansing, for which primarily soaps are used. In this regard, a distinction is made between solid soap, usually in bars, and liquid soap. Accordingly, in a non-limiting embodiment the cosmetic agents are present as shaped bodies that contain surface-active ingredients. In a non-limiting embodiment, the most important ingredients of shaped bodies of this kind are the alkali salts of fatty acids of natural oils and fats, such as having chains of 12 to 18 C atoms. Since lauric acid soaps foam particularly well, coconut and palm kernel oils rich in lauric acid may be included as raw materials for fine soap production. The Na salts of fatty acid mixtures are solid; the K salts are slightly pasty. For saponification, the diluted sodium hydroxide solution or potassium hydroxide solution is added to the fat raw materials in a stoichiometric ratio so that an excess of lye of no more than 0.05% is present in the finished soap. In many instances, soaps today are no longer produced directly from the fats, but from the fatty acids obtained by way of lipolysis. Customary soap additives are fatty acids, fatty alcohols, lanolin, lecithin, vegetable oils, partial glycerides, inter alia, similar fat-like substances for lipid replenishment of the cleansed skin, antioxidants such as ascorbil palmitate or tocopherol for preventing auto-oxidation of the soap (rancidity), complexing agents such as nitrilotriacetate for binding heavy metal traces that could catalyze the auto-oxidative spoilage, perfume oils for achieving the desired fragrance notes, dyes for coloring the bars of soap, and optionally special additives.

Liquid soaps are based on both K salts of natural fatty acids and on synthetic anionic surfactants. In aqueous solution, they contain fewer washing-active substances than solid soaps, and have the customary additives, optionally comprising viscosity-regulating components and pearlescing additives. Due to the convenient and hygienic application from dispensers, they are used in public lavatories and the like. Washing lotions for particularly sensitive skin are based on mild synthetic surfactants comprising additives of skin care substances that are set to a neutral or slightly acidic pH (pH 5.5).

For cleansing primarily facial skin, a number of additional preparations are available, such as facial toners, cleansing lotions, cleansing milks, cleansing creams and cleansing pastes; face packs are used in part for cleansing, but they generally refresh and nourish the facial skin. Facial toners are typically aqueous-alcoholic solutions having a low surfactant content and further skin care substances. Cleansing lotions, milks, creams and pastes are typically based on O/W emulsions that have a relatively low fatty component content and have cleansing and nourishing additives. What are known as scruffing and peeling preparations contain substances that have a mild keratolytic effect to remove the uppermost necrotic layers of dead skin, in part comprising additives of abrasively acting powder. Almond bran, which has long been used as a mild skin cleansing agent, is frequently still a component of preparations of this kind today. Agents for the cleansing treatment of blemished skin also contain antibacterial and anti-inflammatory substances, since the accumulation of sebaceous material in comedones (blackheads) represents a breeding ground for bacterial infections and tends to cause inflammation. The wide range of different skin cleansing products offered varies in terms of the composition and content of different active ingredients depending on the different skin types and specific treatment purposes.

Further cosmetic agents may include those used for influencing body odor. This refers in particular to deodorizing agents. Deodorants of this kind are able to mask, remove or destroy odors. Unpleasant body odors arise from the bacterial decomposition of sweat, in particular in the warm and moist axilla regions, where microorganisms encounter good living conditions. As a result, antimicrobial substances are the most important ingredients of deodorants. In particular, antimicrobial substances that have a substantially selective effectiveness with respect to the bacteria responsible for body odor are preferred. Non-limiting active ingredients, however, have only a bacteriostatic effect and by no means completely destroy the bacterial flora. Antimicrobial agents include in general all suitable preservatives that specifically work against gram-positive bacteria. These are, for example, Irgasan DP 300 (triclosan, 2,4,4′-trichloro-2′-hydroxydiphenyl ether), chlorhexidine (1,1′-hexamethylenebis(5-(4′-chlorophenyl)-biguanide) and 3,4,4′-trichlorocarbanilide. In principle, quaternary ammonium compounds are also suitable. Due to their high antimicrobial effectiveness, all these substances are used only in low concentrations of approximately 0.1 to 0.3 wt. %. Moreover, numerous odorants also exhibit antimicrobial properties. Accordingly, such odorants having antimicrobial properties are used in deodorants. In particular, farnesol and phenoxyethanol should be mentioned in this regard, e.g. when the deodorants include bacteriostatically active odorants. The odorants may be present again in the form of silyl enol ethers. However, it is also possible that precisely these antibacterially active odorants are not used in the form of silyl enol ethers and are then used in mixtures with other odorants which are present as silyl enol ethers. A further group of essential ingredients of deodorants are enzyme inhibitors, which inhibit the enzymatic decomposition of sweat, such as citric acid triethyl ester or zinc glycinate. Furthermore, essential ingredients of deodorants are also antioxidants, which are intended to prevent oxidation of sweat components.

In a further likewise embodiment, the cosmetic agent is a hair setting agent that contains polymers for setting, such as when at least one polyurethane is contained among the polymers.

Finally, air care agents, for example in the form of sprays, and insect repellents which, in addition to the silyl enol ethers described herein, may contain the ingredients which are typical and known for agents of this kind.

In principle, all embodiments disclosed in connection with the silyl enol ethers and the agents are also applicable to the methods and uses described, and vice versa. It is self-evident, for example, that all specific silyl enol ethers described herein are applicable to said agents and methods and can be used as described herein.

### EXAMPLES

### General Method

### Preparation of Silyl Enol Ethers Using Lithium Diisopropylamine (LDA)

Water-free THF (2 mL/mmol ketone) contained in a flask became diisopropylamine (DIPA) (1.1 eq) in a nitrogen atmosphere and the solution was cooled to −78° C. while stirring. Then, n-butyllithium (n-BuLi) was added dropwise using a dropping funnel (10.5 eq) and the reaction mixture was stirred for 15 minutes before slowly being warmed to room temperature. After 10 minutes, the reaction mixture was recooled to −78° C. and a solution of a ketone in water-free THF (0.8 mL/mmol ketone) was added dropwise to the solution. After the addition, the dropping funnel was rinsed with water-free THF (2 mL/mmol ketone) and the reaction mixture was stirred for 1 h before being diluted with water-free THF (2 mL/mmol ketone). The mixture was stirred for an additional 30 minutes and then silyl chloride (1.1 eq) was added dropwise to the reaction mixture. The mixture was kept at −78° C. for 1 h and then slowly warmed to room temperature over 16 h. The reaction was monitored by means of GC-FID and TLC and, as soon as the conversion of the ketone was complete, the reaction mixture was quenched with phosphate buffer (Fisher Scientific, pH 9.0; 2mL/mmol ketone). The product was extracted into methyl-tert-butyl ether (5 mL/mmol ketone) and washed with phosphate buffer (pH 9.0; 3×3 mL/mmol ketone). The organic phase was dried over MgSO₄ and the solvent removed in vacuo followed by purification by means of column chromatography or distillation.

### Preparation of Silyl Enol Ethers Using Lithium Hexamethyldisilazane (LiHMDS)

This method was adapted based on the method described in the literature (Hurlocker et al., Org. Lett., 2014, 16(6), 4280).

Water-free THF (8 mL/mmol ketone) contained in a flask was added to HMDS (hexamethyldisilazane) (1,1 eq) and n-BuLi (1,1 eq) in a nitrogen atmosphere at 0° C. After the addition, the reaction mixture was warmed to room temperature over 15 minutes and then cooled to −78° C. A solution of a ketone in water-free THF (1 mL/mmol ketone) was added dropwise to the solution, and the mixture was stirred for 10 minutes. Silyl chloride (1.1 eq) was then added dropwise to the reaction mixture and the mixture was warmed slowly to room temperature over 16 h. The reaction was monitored by means of GC-FID and TLC and, as soon as the conversion of the ketone was complete, the reaction mixture was quenched with saturated NaHCO₃ solution (8mL/mmol ketone). The product was extracted into methyl tert-butyl ether (3×10 mL/mmol ketone), the organic phases combined and dried over MgSO₄. The solvent was removed in vacuo followed by purification by means of column chromatography or distillation.

### Example 1

### 2-(trimethyl)silyloxy-1-undecene

2-(trimethyl)silyloxy-1-undecene was prepared by means of general method 1.

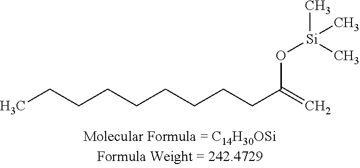 [SMILES: C=C(CCCCCCCCC)O[Si](C)(C)C]

24.3 g (99%, colorless oil)
R_{f}=0.36 (n-Pe)
¹H NMR (CDCl₃, 400 MHz): δ (ppm) 4.05 (s, 2H), 1.99 (t, 2H), 1.48-1.38 (m, 2H), 1.38-1.15 (m, 12H), 0.87 (t, 3H), 0.20 (s, 9H).
MS (ESI+, 150V): 243 [M+H]

Spectroscopic data consistent with the literature [Miura et al., Bull. Chem. Soc. Japan, 1991, 64(5), 1542].

### Example 2

### 2-(triethyl)silyloxy-1-undecene

2-(triethyl)silyloxy-1-undecene was prepared by means of general method 1.

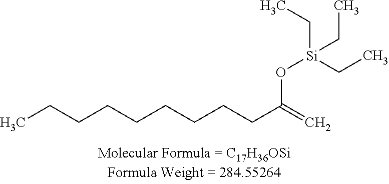 [SMILES: C=C(CCCCCCCCC)O[Si](CC)(CC)CC]

2.90 g (quantitative; colorless oil)
R_{f}=0.66 (1% Et₃N in Et₂O/n-Hex, 9:1)
¹H NMR (CDCl₃, 400 MHz): δ (ppm) 4.01 (d, 2H), 2.02 (t, 2H), 1.47 (tt, 2H), 1.37-1.20 (m, 12H), 0.98 (t, 9H), 0.89 (t, 3H), 0.68 (q, 6H).
¹³C NMR (CDCl₃, 100 MHz): δ (ppm) 159.9, 89.1, 36.6, 32.0, 29.6, 29.6, 29.4, 29.2, 27.0, 22.7, 14.1, 6.8, 5.0.
MS (ESI+, 150V): 285 [M+H]
IR (neat, cm⁻¹): \tilde{v}=2924 (s), 2855 (m), 1655 (w), 1613 (w), 1459 (m), 1272 (m), 1239 (m), 1111 (w), 1006 (s), 809 (m), 729 (s).

### Example 3

### 2-(trimethyl)silyloxy-4-phenyl-1-butene

2-(trimethyl)silyloxy-4-phenyl-1-butene was prepared by means of general method 1.

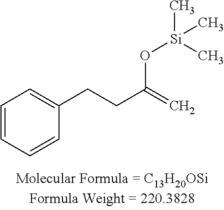

10.82 g (98%, colorless oil)
R_{f}=0.21 (n-Pe)
R_{f}=0.68 (MTBE)
¹H NMR (CDCl₃, 400 MHz): δ (ppm) 7.08 (m, 2H), 6.99 (m, 3H), 3.88 (s, 2H), 2.59 (t, 2H), 2.14 (t, 2H), 0.03 (m, 9H).
MS (ESI+, 150V): 221 [M+H]
Spectroscopic data consistent with the literature [Haraguchi et al., Tetrahedron, 2015, 71(49), 8830].

### Example 4

### 2-(triethyl)silyloxy-4-phenyl-1-butene

2-(triethyl)silyloxy-4-phenyl-1-butene was prepared by means of general method 1.

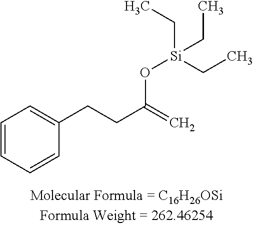 [SMILES: C=C(CCc1ccccc1)O[Si](CC)(CC)CC]

1.24 g (47%; colorless oil)
R_{f}=0.72 (1% Et₃N in MTBE/n-Hex, 1:1)
¹H NMR (CDCl₃, 400 MHz): δ (ppm) 7.27 (ddd, 2H), 7.21-7.17 (m, 3H), 4.03 (d, 2H), 2.80 (t, 2H), 2.36 (t, 2H), 0.99 (t, 9H), 0.72 (q, 6H)
¹³C NMR (CDCl₃, 100 MHz): δ (ppm) 158.9, 141.9, 128.4, 128.3, 125.8, 89.6, 38.5, 33.5, 6.8, 5.0.
IR (ATR, cm⁻¹): v=2955 (w), 2877 (w), 1633 (w), 1455 (w), 1291 (w), 1273 (w), 1239 (w), 1002 (m), 727 (s), 696 (s).
Spectroscopic data consistent with the literature [Hurlocker et al., Org. Left., 2014, 16(6), 4280].

### Example 5

### (4-phenyl-2-(tert-butyldimethylsilyl)oxy-1-butene) & (4-phenyl-2-(tert-butyldimethylsilyl)oxy-2-butene)

(4-phenyl-2-(tert-butyldimethylsilyl)oxy-1-butene) & (4-phenyl-2-(tert-butyldimethylsilyl)oxy-2-butene) were prepared by means of general method 2.

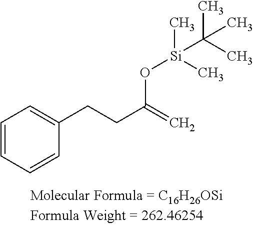 [SMILES: C=C(CCc1ccccc1)O[Si](C)(C)C(C)(C)C]

R_{f} (1% Et₃N (v/v) in Et₂O/ⁿHex, 1:9)=0.59

1-ene: (4-phenyl-2-(tert-butyldimethylsilyl)oxy-1-butene)
¹H NMR (CDCl₃) −7.31-7.12 (m, 5H), 4.04 (s, 2H), 2.80 (q, 2H), 2.33 (q, 2H), 1.01-0.90 (m, 9H), 0.20-0.10 (m, 6H).
¹³C NMR (CDCl₃) −158.8, 141.9, 128.5, 128.3, 125.7, 90.1, 38.6, 33.4, 25.8, 18.1, −4.7.

2-ene: (Isomer a) (4-phenyl-2-(tert-butyldimethylsilyl)oxy-2-butene; (E)/(Z))
¹H NMR (CDCl₃) −7.31-7.12 (m, 5H), 4.85 (t, 1H), 3.39 (d, 2H), 1.83 (s, 3H), 1.01-0.90 (m, 9H), 0.20-0.10 (m, 6H).
¹³C NMR (CDCl₃) −149.2, 141.9, 128.4, 128.3, 125.7, 106.6, 33.4, 25.7, 17.8, 18.0, −4.4.

2-ene: (Isomer b) (4-phenyl-2-(tert-butyldimethylsilyl)oxy-2-butene; (E)/(Z))
¹H NMR (CDCl₃) −7.31-7.12 (m, 5H), 4.60 (t, 1H), 3.47 (d, 2H), 1.83 (s, 3H), 1.01-0.90 (m, 9H), 0.20-0.10 (m, 6H).
¹³C NMR (CDCl₃) −147.4, 142.3, 128.4, 128.2, 125.5, 107.1, 31.6, 25.8, 22.8, 18.1, −3.7.

Spectroscopic data consistent with the literature [Haraguchi et al., Tetrahedron, 2015, 71(49), 8830].

### Example 6

### Odor Test for Silyl Enol Ethers of Methyl Nonyl Ketone

### Compounds Used:

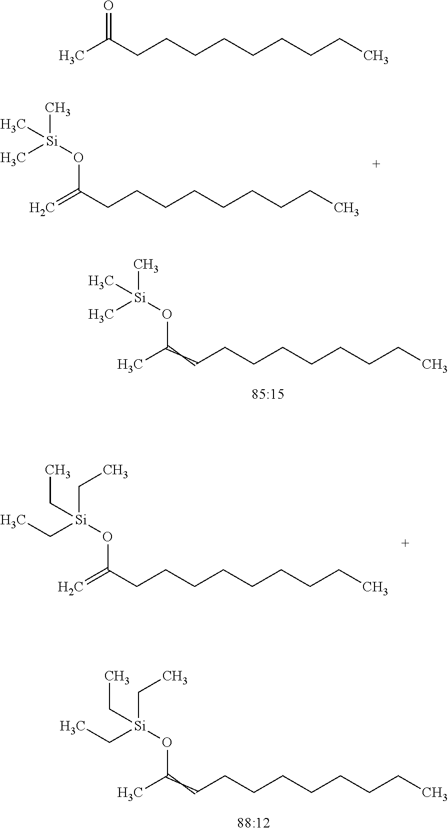

### Reagents:

| | Concentration | Concentration |
|---|---|---|
| Compounds | mg mL⁻¹ | mM |
| 2-undecanone (methylnonyl ketone; MNK) | 35.5 mg (EtOH) | 208.5 |
| MW = 170.29 g mol⁻¹, ρ = 0.825 g mL⁻¹ | 36.9 mg (Et₂O) | 216.7 |
| [112-12-9] Sigma Aldrich | | |
| 2-(trimethyl)silyloxy-1-undecene/ | 51.4 mg (EtOH) | 212.0 |
| 2-(trimethyl)silyloxy-2-undecene | 48.6 mg (Et₂O) | 200.4 |
| (85:15)^{a} (MNK-TMS) | | |
| MW = 242.47 g mol⁻¹ | | |
| 2-(triethyl)silyloxy-1-undecene/ | 58.3 mg (EtOH) | 204.9 |
| 2-(triethyl)silyloxy-2-undecene | 57.9 mg (Et₂O) | 203.5 |
| (88:12)^{a} (MNK-TES) | | |
| MW = 284.55 g mol⁻¹ | | |
| Phosphate buffer, pH 3.0 | | |
| Prod. no. 03082.3000 Bernd Kraft | | |

^{a}determined by means of ¹H NMR

### Method

The values given are average values of two test persons. The samples were prepared as 200 mM solutions in either EtOH or Et₂O. The solution was absorbed on odor test strips. After the strips were soaked in the solution, they were dried for 10 minutes, then sprayed with pH 3.0 buffer and smelled after the following times:
A—after 10 min (dry)
B—immediately after spraying with buffer
C—30 min after spraying
D—1 hour after spraying
E—2 hours after spraying
F—4 hours after spraying
G—19.5 hours after spraying
H—24 hours after spraying
I—48 hours after spraying
J—72 hours after spraying
K—6 days after spraying

The controls were not sprayed with buffer, but instead smelled after the specified time without having been previously sprayed. “Activated” means that the corresponding sample was sprayed with buffer. The odor intensity is evaluated on a scale from 0 (no odor) to 6 (very strong). The results are shown in Table 1.

TABLE 1

| | A | B | C | D | E | F | G | H | I |
|---|---|---|---|---|---|---|---|---|---|
| 200 mM in Et₂O | | | | | | | | | |
| MNK (control) | 3.0 | 3.0 | 4.0 | 4.0 | 4.0 | 2.5 | 0.2 | 0.5 | 0.2 |
| MNK-TES (activated) | 2.2 | 2.5 | 1.5 | 1.0 | 1.5 | 1.2 | 0.8 | 0.6 | 0.3 |
| 200 mM in EtOH | | | | | | | | | |
| MNK (control) | 4.0 | 4.0 | 4.5 | 4.5 | 4.0 | 1.2 | 0.0 | 0.2 | 0.0 |
| MNK-TMS (control) | 2.0 | 2.0 | 2.0 | 1.5 | 2.5 | 0.7 | 0.7 | 0.2 | 0.1 |
| MNK-TES (control) | 1.5 | 1.5 | 2.5 | 2.0 | 2.0 | 1.8 | 0.2 | 0.3 | 0.05 |
| MNK (activated) | 4.0 | 4.0 | 3.5 | 3.0 | 4.0 | 1.8 | 0.2 | 0.2 | 0.3 |
| MNK-TES (activated) | 1.5 | 2.0 | 1.5 | 1.0 | 1.5 | 1.2 | 0.5 | 0.5 | 0.5 |

### Example 7

### Odor Test for Silyl Enol Ethers of Benzylacetone

### Compounds Used:

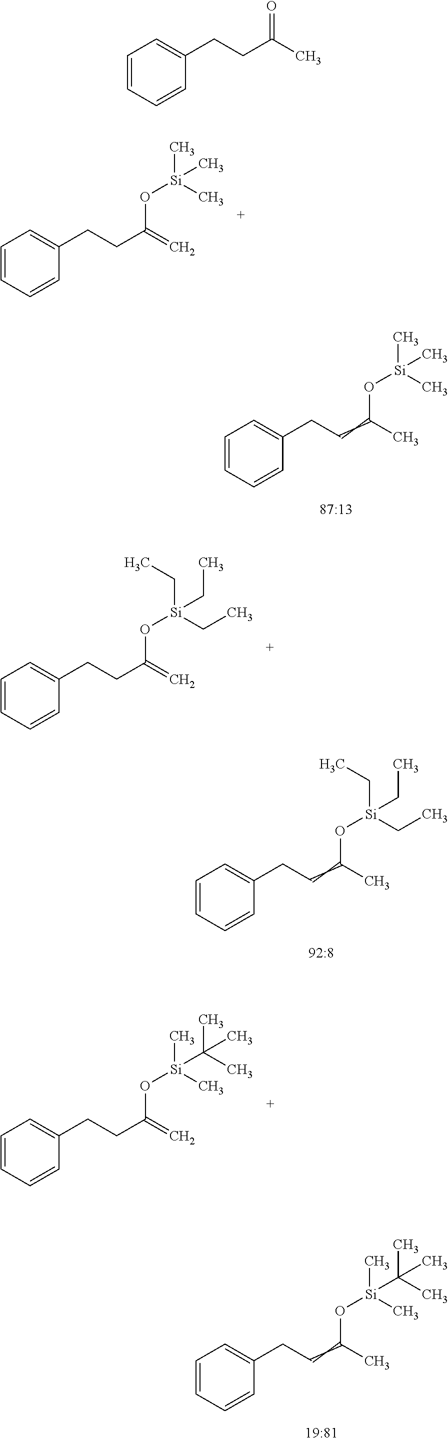

### Reagents:

| Compound | Mass/mL | Concentration |
|---|---|---|
| 4-phenyl-2-butanone (benzylacetone; BA) | 30.3 mg | 204.5 |
| MW = 148.40 g mol⁻¹, ρ = 0.989 g mL⁻¹ | (EtOH) | |
| [2550-26-7] Alfa Aesar | | |
| 2-(trimethyl)silyloxy-4-phenyl-1-butene/ | 46.6 mg | 211.5 |
| 2-(trimethyl)silyloxy-4-phenyl-2-butene | (EtOH) | |
| (87:13)^{a} (BA-TMS) | | |
| MW = 220.38 g mol⁻¹ | | |
| 2-(triethyl)silyloxy-4-phenyl-1-butene/ | 56.8 mg | 216.4 |
| 2-(triethyl)silyloxy-4-phenyl-2-butene | (EtOH) | |
| (92:8)^{a} (BA-TES) | | |
| MW = 262.46 g mol⁻¹ | | |
| 2-(tert-butyldimethyl)silyloxy-4-phenyl-1-butene/ | 54.4 mg | 207.3 |
| 2-(tert-butyldimethyl)silyloxy-4-phenyl-2-butene | (EtOH) | |
| (19:81)^{a} (BA-TBDMS) | | |
| MW = 262.46 g mol⁻¹ | | |
| Phosphate buffer, pH 3.0 | | |
| Prod.no. 03082.3000 Bernd Kraft | | |

^{a}determined by means of ¹H NMR

### Method

The test was carried out as described in Example 6. The results are shown in Table 2.

TABLE 2

| | A | B | C | D | E | F | G | H | I | J | K |
|---|---|---|---|---|---|---|---|---|---|---|---|
| 200 mM in EtOH | | | | | | | | | | | |
| BA (control) | 5.0 | 5.0 | 4.5 | 4.5 | 4.5 | 3.0 | 1.8 | 1.0 | 1.0 | 0.8 | 0.5 |
| BA-TMS (control) | 2.5 | 2.5 | 3.0 | 2.0 | 2.5 | 2.5 | 0.5 | 0.8 | 0.8 | 1.0 | 1.0 |
| BA-TBDMS (control) | 1.0 | 1.0 | 2.0 | 0.5 | 1.5 | 0.5 | 1.0 | 1.0 | 1.8 | 2.0 | 1.8 |
| BA (activated) | 5.0 | 4.5 | 3.0 | 3.0 | 4.0 | 3.5 | 2.2 | 1.8 | 1.3 | 1.0 | 0.3 |
| BA-TMS (activated) | 2.5 | 2.5 | 2.0 | 1.2 | 2.2 | 1.8 | 1.5 | 1.8 | 1.5 | 1.5 | 1.0 |
| BA-TBDMS (activated) | 1.0 | 1.0 | 1.5 | 1.0 | 1.5 | 1.2 | 2.0 | 1.2 | 1.8 | 2.0 | 1.2 |

### Example 8

### Boost of Silyl Enol Ethers of Methylnonyl Ketone

The compounds were also tested for their odor-increasing effect (“boost effect”).

| | Concentration | Concentration |
|---|---|---|
| Compound | mg mL⁻¹ | mM |
| MNK | 35.5 mg (EtOH) | 208.5 |
| | 36.9 mg (Et₂O) | 216.7 |
| MNK-TMS | 51.4 mg (EtOH) | 212.0 |
| | 48.6 mg (Et₂O) | 200.4 |
| MNK-TES | 58.3 mg (EtOH) | 204.9 |
| | 57.9 mg (Et₂O) | 203.5 |

The compounds were divided into 3 batches:
Control (not sprayed with buffer) after 1, 4 and 5 days
Boost24 (sprayed with buffer after 24 h drying in the fume cupboard)
Boost96 (sprayed with buffer after 4 days drying in the fume cupboard)

The results were evaluated as in Examples 6 and 7 and the results are shown in Table 3.

TABLE 3

| | | Condition | | | | |
|---|---|---|---|---|---|---|
| | | Control | Control | Control | | |
| | | (1 day) | (4 days) | (5 days) | Boost24 | Boost96 |
| | | Day # | | | | |
| | | 1 | 4 | 5 | 2 | 4 |
| | | A | B | C | D | E |
| EtOH | MNK | 0.5 | 0 | 0 | 0.85 | 0.5 |
| | MNK-TMS | 0.75 | 0.5 | 0.25 | 1 | 0.5 |
| | MNK-TES | 0.25 | 0.1 | 0.1 | 2 | 0.25 |
| Et₂O | MNK | 1 | 0 | 0 | 1 | 0 |
| | MNK-TMS | 1 | 0.75 | 0.25 | 1.25 | 0.25 |
| | MNK-TES | 0.25 | 0 | 0 | 2 | 0 |

### Example 9

### Boost of Silyl Enol Ethers of Benzylacetone

The silyl enol ethers of benzylacetone were also tested, similarly to Example 8, for their boost effect.

| | Concentration | Concentration |
|---|---|---|
| Compound | mg mL⁻¹ | mM |
| BA | 29.3 mg (Et₂O) | 206.5 |
| BA-TBDMS | 53.4 mg (Et₂O) | 206.9 |

The compounds were divided into 3 batches:
Control (not sprayed with buffer) after 1.4 and 5 days
Boost24 (sprayed with buffer after 24 h drying in the fume cupboard)
Boost96 (sprayed with buffer after 4 days drying in the fume cupboard)

The results were evaluated as in Examples 6 and 7 and the results are shown in Table 4.

TABLE 4

| | | Condition | | | | |
|---|---|---|---|---|---|---|
| | | Control | Control | Control | | |
| | | (24 hours) | (4 days) | (5 days) | Boost24 | Boost96 |
| | | Day # | | | | |
| | | 1 | 4 | 5 | 2 | 4 |
| Et₂O | BA | 1.25 | 0.5 | 0.1 | 2.25 | 1.75 |
| | BA-TBDMS | 1 | 2 | 1.75 | 2.5 | 1.75 |

1. A compound comprising a silyl enol ether of formula (I): 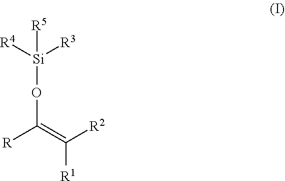
wherein
R, R¹, and R² are independently selected from H, straight-chain or branched, saturated or unsaturated, substituted or unsubstituted hydrocarbon functional groups having 1 to 20 carbon atoms and optionally up to 6 heteroatoms; wherein the heteroatoms are selected from O, S, and N; or R and R¹ or R and R² can be combined with one another in order to form a cyclic group selected from substituted or unsubstituted aryl having up to 20, carbon atoms, substituted or unsubstituted heteroaryl having up to 20, carbon atoms, and 1 to 6 heteroatoms selected from O, S, and N; substituted or unsubstituted cycloalkyl or cycloalkenyl having up to 20, carbon atoms; and substituted or unsubstituted heterocycloalkyl or heterocycloalkenyl having up to 20carbon atoms and 1 to 6heteroatoms selected from O, S, and N;
wherein at least one of R, R¹, and R² is not H; and wherein the functional group —O—CR═CR¹R² is derived from an odorant ketone or odorant aldehyde of formula R—C(O)—CHR¹R²; and
R³, R⁴ and R⁵ are independently selected from straight-chain or branched, saturated or unsaturated, substituted or unsubstituted hydrocarbon functional groups having 1 to 20 carbon atoms.

2. The compound according to claim 1, wherein R is a linear or branched, substituted or unsubstituted, alkyl, alkenyl, or alkynyl functional group having up to 20 carbon atoms.

3. The compound according to claim 1, wherein
(a) one of R¹ or R² is H and wherein the other of R¹ or R² is a straight-chain or branched, saturated or unsaturated, substituted or unsubstituted hydrocarbon functional group having 1 to 20 carbon atoms and optionally up to 6 heteroatoms; or
(b) R¹ and R² are H.

4. The compound according to claim 1, wherein R¹ and R² are H; and wherein R is an alkyl functional group having up to 12 carbon atoms.

5. The compound according to claim 1, wherein the odorant ketone or odorant aldehyde of the formula R—C(O)—CHR¹R² is selected from the group consisting of: adoxal (2,6,10-trimethyl-9-undecenal), cymene (3-(4-isopropylphenyl)-2-methylpropanal), Florhydral (3-(3-isopropylphenyl)butanal), Helional (3-(3,4-methylenedioxyphenyl)-2-methylpropanal), hydroxycitronellal, lauraldehyde, Lyral (3- and 4-(4-hydroxy-4-methylpentyl)-3-cyclohexene-1-carboxaldehyde), methylnonylacetaldehyde, Lilial (3-(4-tert-butylphenyl)-2-methylpropanal), phenylacetaldehyde, undecylenealdehyde, 2,6,10-trimethyl-9-undecenal, 3-dodecen-1-al, melonal (2,6-dimethyl-5-heptenal), 2,4-dimethyl-3-cyclohexene-1-carboxaldehyde (Triplal), 3-(4-tert-butylphenyl)-propanal, 2-methyl-3-(para-methoxyphenyl)propanal, 2-methyl-4-(2,6,6-trimethyl-2(1)-cyclohexen-1-yl)butanal, cis-/trans-3,7-dimethyl-2,6-octadien-1-al, 3,7-dimethyl-6-octen-1-al, [(3,7-dimethyl-6-octenyl)oxy]acetaldehyde, 1,2,3,4,5,6,7,8-octahydro-8,8-dimethyl-2-naphthaldehyde, 2,4-dimethyl-3-cyclohexene-1-carboxaldehyde, 2-methyl-3-(isopropylphenyl)propanal, 1-decanal, 2,6-dimethyl-5-heptenal, 4-(tricyclo[5.2.1.0(2,6)]-decylidene-8)-butanal, octahydro-4,7-methane-1H-indenecarboxaldehyde, alpha-methyl-3,4-(methylenedioxy)-hydrocinnamaldehyde, m-cymene-7-carboxaldehyde, alpha-methylphenylacetaldehyde, 7-hydroxy-3,7-dimethyloctanal, undecenal, 2,4,6-trimethyl-3-cyclohexene-1-carboxaldehyde, 4-(3)(4-methyl-3-pentenyl)-3-cyclohexenecarboxaldehyde, 1-dodecanal, 2,4-dimethylcyclohexene-3-carboxaldehyde, 4-(4-hydroxy-4-methylpentyl)-3-cyclohexene-1-carboxaldehyde, 7-methoxy-3,7-dimethyloctan-1-al, 2-methylundecanal, 2-methyldecanal, 1-nonanal, 1-octanal, 2,6,10-trimethyl-5,9-undecadienal, 2-methyl-3-(4-tert-butyl)propanal, dihydrocinnamaldehyde, 1-methyl-4-(4-methyl-3-pentenyl)-3-cyclohexene-1-carboxaldehyde, 5- or 6-methoxy-hexahydro-4,7-methanindan-1- or 2-carboxaldehyde, 3,7-dimethyloctan-1-al, 1-undecanal, 10-undecen-1-al, 1-methyl-3-(4-methylpentyl)-3-cyclohexenecarboxaldehyde, 7-hydroxy-3,7-dimethyl-octanal, trans-4-decenal, 2,6-nonadienal, para-tolylacetaldehyde, 4-methylphenylacetaldehyde, 2-methyl-4-(2,6,6-trimethyl-1-cyclohexen-1-yl)-2-butenal, 3,5,6-trimethyl-3-cyclohexenecarboxaldehyde, 3,7-dimethyl-2-methylene-6-octenal, phenoxyacetaldehyde, 5,9-dimethyl-4,8-decadienal, peonyaldehyde (6,10-dimethyl-3-oxa-5,9-undecadien-1-al), hexahydro-4,7-methanindane-1-carboxaldehyde, 2-methyloctanal, alpha-methyl-4-(1-methylethyl)benzeneacetaldehyde, 6,6-dimethyl-2-norpinen-2-propionaldehyde, para-methylphenoxyacetaldehyde, 3,5,5-trimethylhexanal, hexahydro-8,8-dimethyl-2-naphthaldehyde, 3-propylbicyclo[2.2.1]-hept-5-en-2-carbaldehyde, 9-decenal, 3-methyl-5-phenyl-1-pentanal, methylnonylacetaldehyde, hexanal, trans-2-hexenal, 2-undecanone (methylnonyl ketone), methyl-beta-naphthyl ketone, musk indanone (1,2,3,5,6,7-hexahydro-1,1,2,3,3-pentamethyl-4H-inden-4-one), tonalide (6-acetyl-1,1,2,4,4,7-hexamethyltetralin), alpha-damascone, beta-damascone, delta-damascone, iso-damascone, damascenone, methyl dihydrojasmonate, menthone, carvone, camphor, Koavone (3,4,5,6,6-pentamethylhept-3-en-2-one), fenchone, alpha-ionone, beta-ionone, gamma-methyl-ionone, fleuramone (2-heptylcyclopentanone), dihydrojasmone, cis-jasmone, 1-(1,2,3,4,5,6,7,8-octahydro-2,3,8,8-tetramethyl-2-naphthalenyl)-ethan-1-one and isomers thereof, methyl cedrenyl ketone, acetophenone, methyl acetophenone, para-methoxy acetophenone, methyl-beta-naphthyl ketone, benzylacetone, para-hydroxyphenyl butanone, celery ketone (3-methyl-5-propyl-2-cyclohexenone), 6-isopropyldeca-hydro-2-naphthone, dimethyloctenone, frescomenthe (2-butan-2-ylcyclohexan-1-one), 4-(1-ethoxyvinyl)-3,3,5,5-tetramethylcyclohexanone, methylheptenone, 2-(2-(4-methyl-3-cyclohexen-1-yl)propyl)cyclopentanone, 1-(p-menthene-6(2)yl)-1-propanone, 4-(4-hydroxy-3-methoxyphenyl)-2-butanone, 2-acetyl-3,3-dimethylnorbornane, 6,7-dihydro-1,1,2,3,3-pentamethyl-4(5H)indanone, 4-damascol, Dulcinyl (4-(1,3-benzodioxo1-5-yl)butan-2-one), hexalone (1-(2,6,6-trimethyl-2-cyclohexen-1-yl)-1,6-heptadien-3-one), isocyclemone E (2-acetonaphthone-1,2,3,4,5,6,7,8-octahydro-2,3,8,8-tetramethyl), methylnonyl ketone, methylcyclocitrone (1-(3,5,6-trimethyl-1-cyclohex-3-enyl)ethanone), methylene lavender ketone (3-hydroxymethylnonan-2-one), Orivone (4-tert-amylcyclohexanone), 4-tert-butylcyclohexanone, delphone (2-pentyl-cyclopentanone), muscone (CAS 541-91-3), neobutenone (1-(5,5-dimethyl-1-cyclo-hexenyl)pent-4-en-1-one), plicatone (CAS 41724-19-0), veloutone (2,2,5-trimethyl-5-pentylcyclopentan-1-one), 2,4,4,7-tetramethyl-oct-6-en-3-one and tetramerane (6,10-dimethylundecen-2-one).

6. The compound according to claim 1, wherein R³, R⁴, and R⁵ are C₁₋₆ hydrocarbons.

7. (canceled)

8. A composition comprising:
an agent, selected from the group comprising a washing agent, a cleaning agent, a cosmetic agent, an air care agent, an insect repellent, or combinations thereof; and
the silyl enol ether compound according to claim 1.

9. The composition according to claim 8, wherein the agent is
(a) a liquid or gel agent
(b) a powdery or granular agent;
(c) an agent in the form of shaped bodies; or a cosmetic agent for treating hair or skin; and
(e) combinations thereof.

10. The composition according to claim 8, wherein the silyl enol ether compound is present in the composition in an amount ranging from 0.001 to 50 wt. % based on the total composition.

11. The composition according to claim 8, wherein R of the compound is a linear or branched, substituted or unsubstituted, alkyl, alkenyl, or alkynyl functional group having up to 20 carbon atoms.

12. The composition according to claim 8, wherein
(a) one of R¹ or R² of the compound is H and wherein the other of R¹ or R² is a straight-chain or branched, saturated or unsaturated, substituted or unsubstituted hydrocarbon functional group having 1 to 20 carbon atoms and optionally up to 6 heteroatoms; or
(b) R¹ and R² are H.

13. The composition according to claim 8, wherein R¹ and R² of the compound are H and R is an alkyl functional group having up to 12 carbon atoms.

14. The composition according to claim 8, wherein the odorant ketone or odorant aldehyde of the formula R—C(O)—CHR¹R² is selected from the group consisting of: adoxal (2,6,10-trimethyl-9-undecenal), cymene (3-(4-isopropylphenyl)-2-methylpropanal), Florhydral (3-(3-isopropylphenyl)butanal), Helional (3-(3,4-methylenedioxyphenyl)-2-methylpropanal), hydroxycitronellal, lauraldehyde, Lyral (3- and 4-(4-hydroxy-4-methylpentyl)-3-cyclohexene-1-carboxaldehyde), methylnonylacetaldehyde, Lilial (3-(4-tert-butylphenyl)-2-methylpropanal), phenylacetaldehyde, undecylenealdehyde, 2,6,10-trimethyl-9-undecenal, 3-dodecen-1-al, melonal (2,6-dimethyl-5-heptenal), 2,4-dimethyl-3-cyclohexene-1-carboxaldehyde (Triplal), 3-(4-tert-butylphenyl)-propanal, 2-methyl-3-(para-methoxyphenyl)propanal, 2-methyl-4-(2,6,6-trimethyl-2(1)-cyclohexen-1-yl)butanal, cis-trans-3,7-dimethyl-2,6-octadien-1-al, 3,7-dimethyl-6-octen-1-al, [(3,7-dimethyl-6-octenyl)oxy]acetaldehyde, 1,2,3,4,5,6,7,8-octahydro-8,8-dimethyl-2-naphthaldehyde, 2,4-dimethyl-3-cyclohexene-1-carboxaldehyde, 2-methyl-3-(isopropylphenyl)propanal, 1-decanal, 2,6-dimethyl-5-heptenal, 4-(tricyclo[5.2.1.0(2,6)]-decylidene-8)-butanal, octahydro-4,7-methane-1H-indenecarboxaldehyde, alpha-methyl-3,4-(methylenedioxy)-hydrocinnamaldehyde, m-cymene-7-carboxaldehyde, alpha-methylphenylacetaldehyde, 7-hydroxy-3,7-dimethyloctanal, undecenal, 2,4,6-trimethyl-3-cyclohexene-1-carboxaldehyde, 4-(3)(4-methyl-3-pentenyl)-3-cyclohexenecarboxaldehyde, 1-dodecanal, 2,4-dimethylcyclohexene-3-carboxaldehyde, 4-(4-hydroxy-4-methylpentyl)-3-cyclohexene-1-carboxaldehyde, 7-methoxy-3,7-dimethyloctan-1-al, 2-methylundecanal, 2-methyldecanal, 1-nonanal, 1-octanal, 2,6,10-trimethyl-5,9-undecadienal, 2-methyl-3-(4-tert-butyl)propanal, dihydrocinnamaldehyde, 1-methyl-4-(4-methyl-3-pentenyl)-3-cyclohexene-1-carboxaldehyde, 5- or 6-methoxy-hexahydro-4,7-methanindan-1- or 2-carboxaldehyde, 3,7-dimethyloctan-1-al, 1-undecanal, 10-undecen-1-al, 1-methyl-3-(4-methylpentyl)-3-cyclohexenecarboxaldehyde, 7-hydroxy-3,7-dimethyl-octanal, trans-4-decenal, 2,6-nonadienal, para-tolylacetaldehyde, 4-methylphenylacetaldehyde, 2-methyl-4-(2,6,6-trimethyl-1-cyclohexen-1-yl)-2-butenal, 3,5,6-trimethyl-3-cyclohexenecarboxaldehyde, 3,7-dimethyl-2-methylene-6-octenal, phenoxyacetaldehyde, 5,9-dimethyl-4,8-decadienal, peonyaldehyde (6,10-dimethyl-3-oxa-5,9-undecadien-1-al), hexahydro-4,7-methanindane-1-carboxaldehyde, 2-methyloctanal, alpha-methyl-4-(1-methylethyl)benzeneacetaldehyde, 6,6-dimethyl-2-norpinen-2-propionaldehyde, para-methylphenoxyacetaldehyde, 3,5,5-trimethylhexanal, hexahydro-8,8-dimethyl-2-naphthaldehyde, 3-propylbicyclo[2.2.1]-hept-5-en-2-carbaldehyde, 9-decenal, 3-methyl-5-phenyl-1-pentanal, methylnonylacetaldehyde, hexanal, trans-2-hexenal, 2-undecanone (methylnonyl ketone), methyl-beta-naphthyl ketone, musk indanone (1,2,3,5,6,7-hexahydro-1,1,2,3,3-pentamethyl-4H-inden-4-one), tonalide (6-acetyl-1,1,2,4,4,7-hexamethyltetralin), alpha-damascone, beta-damascone, delta-damascone, iso-damascone, damascenone, methyl dihydrojasmonate, menthone, carvone, camphor, Koavone (3,4,5,6,6-pentamethylhept-3-en-2-one), fenchone, alpha-ionone, beta-ionone, gamma-methyl-ionone, fleuramone (2-heptylcyclopentanone), dihydrojasmone, cis-jasmone, 1-(1,2,3,4,5,6,7,8-octahydro-2,3,8,8-tetramethyl-2-naphthalenyl)-ethan-1-one and isomers thereof, methyl cedrenyl ketone, acetophenone, methyl acetophenone, para-methoxy acetophenone, methyl-beta-naphthyl ketone, benzylacetone, para-hydroxyphenyl butanone, celery ketone (3-methyl-5-propyl-2-cyclohexenone), 6-isopropyldeca-hydro-2-naphthone, dimethyloctenone, frescomenthe (2-butan-2-ylcyclohexan-1-one), 4-(1-ethoxyvinyl)-3,3,5,5-tetramethylcyclohexanone, methylheptenone, 2-(2-(4-methyl-3-cyclohexen-1-yl)propyl)cyclopentanone, 1-(p-menthene-6(2)yl)-1-propanone, 4-(4-hydroxy-3-methoxyphenyl)-2-butanone, 2-acetyl-3,3-dimethylnorbornane, 6,7-dihydro-1,1,2,3,3-pentamethyl-4(5H)indanone, 4-damascol, Dulcinyl (4-(1,3-benzodioxol-5-yl)butan-2-one), hexalone (1-(2,6,6-trimethyl-2-cyclohexen-1-yl)-1,6-heptadien-3-one), isocyclemone E (2-acetonaphthone-1,2,3,4,5,6,7,8-octahydro-2,3,8,8-tetramethyl), methylnonyl ketone, methylcyclocitrone (1-(3,5,6-trimethyl-1-cyclohex-3-enyl)ethanone), methylene lavender ketone (3-hydroxymethylnonan-2-one), Orivone (4-tert-amylcyclohexanone), 4-tert-butylcyclohexanone, delphone (2-pentyl-cyclopentanone), muscone (CAS 541-91-3), neobutenone (1-(5,5-dimethyl-1-cyclo-hexenyl)pent-4-en-1-one), plicatone (CAS 41724-19-0), veloutone (2,2,5-trimethyl-5-pentylcyclopentan-1-one), 2,4,4,7-tetramethyl-oct-6-en-3-one and tetramerane (6,10-dimethylundecen-2-one).

15. The composition according to claim 8, wherein R³, R⁴, and R⁵ of the silyl enol ether compound are C₁₋₆ hydrocarbons.

16. The composition according to claim 8, wherein R, R1, and R2 of the silyl enol ether compound are independently selected from a straight-chain or branched, saturated or unsaturated, substituted or unsubstituted hydrocarbon functional groups having 1 to 12 carbon atoms and optionally up to 6 heteroatoms.

17. The composition according to claim 16, wherein R, R1, and R2 of the silyl enol ether compound are independently selected from a straight-chain or branched, saturated or unsaturated, substituted or unsubstituted hydrocarbon functional groups having 1 to 12 carbon atoms and optionally up to 4 heteroatoms.

18. The composition according to claim 8, wherein R is selected from methyl, ethyl, propyl, butyl, pentyl, hexyl, heptyl, octyl, nonyl, or decyl.

19. The composition according to claim 15, wherein R³, R⁴, and R⁵ of the silyl enol ether compound are independently selected from methyl or ethyl.
